(19) United States
(12) Patent Application Publication
(10) Pub. No.: US 2008/0109257 A1
(43) Pub. Date: May 8, 2008

(54) Systems and methods for a holistic well-being assessment

(76) Inventor: Henry Albrecht, Bellevue, WA (US)

Correspondence Address: BLACK LOWE & GRAHAM, PLLC, 701 FIFTH AVENUE, SUITE 4800, SEATTLE, WA, 98104, US

(21) Appl. No.: 11/879,030

(22) Filed: Jul. 12, 2007

Related U.S. Application Data
(60) Provisional application No. 60/807,178, filed on Jul. 12, 2006.
(60) Provisional application No. 60/938,996, filed on May 18, 2007.

(51) Int. Cl. G06Q 50/00; A61B 5/00

(52) U.S. Cl. 705/2; 600/300

(57) ABSTRACT

Systems and methods are described for an assessment tool built on a holistic well-being improvement model. The model consists of a series of assessments including, but not limited to a well-being improvement assessment, work productivity assessment, and a clinical health risk assessment. A member would complete these assessments through an internet site, on paper, on the phone, and/or on any other medium generally known in the art. After a member participates in the assessment, the assessment is analyzed based on a series of predefined algorithms. A member is then provided with system tools and access to third party behavior change tools and resources. Concurrently the data is compiled for the benefit of an employer and/or a provider in the form of an aggregate report in order to understand the status of the current population's well being and also providing the ability to predict future outcomes. The system further tracks the progress of a member on their well-being goals and behaviors.

### RELATED APPLICATIONS

This application claims the benefit of and priority to and incorporates by reference in its totality U.S. Provisional Application Ser. No. 60/938,996 filed May 18, 2007. This application claims the benefit of and priority to and incorporates by reference in its totality U.S. Provisional Application Ser. No. 60/807,178 filed Jul. 12, 2006. All of the foregoing applications are hereby incorporated by reference in their entirety as if fully set forth herein.

### COPYRIGHT NOTICE

This disclosure is protected under United States and International Copyright Laws. © 2006-2007 Limeade, Inc. All Rights Reserved. A portion of the disclosure of this patent document contains material which is subject to copyright protection. The copyright owner has no objection to the facsimile reproduction by anyone of the patent document or the patent disclosure after formal publication by the U.S. Patent and Trademark Office, as it appears in the U.S. Patent and Trademark Office patent file or records, but otherwise reserves all copyright rights whatsoever.

### BACKGROUND OF THE INVENTION

Individuals are not as happy or fulfilled as they want to be. They lack a sense of overall well-being. According to proprietary research, few people surveyed are happy with themselves “just as they are.” Many people suffer from a lack of well-being of various types, e.g., physical or emotional. Many organizations lack a big-picture understanding of what areas of life, if improved; have the best likelihood of improving well-being.

An organization is made up of a population of individual people. One type of organization in the United States is an employer. A majority of employers face constant increases in health care costs and lack a method for managing the “health” and “well being” of their employees.

Employers have a vested interest in having an engaged, productive workforce. An individual's well-being is directly related to their productivity. As an example, an employee in poor physical shape may be more prone to sickness, and therefore be more likely to use a large amount of sick days. An employer would be interested to learn how many employees are not in shape, and possible solutions for those people. The employer is looking to identify a problem, be given a recommendation for a solution, and then execute the solution that can result in the greatest good.

An employer may attempt to implement a program to encourage smoking cessation, curb alcoholism or promote healthy eating. While these programs may be a step in the right direction, they often lack engagement from employees, and, further, employers are not provided with any metrics to test overall success rates (e.g., reduction in health care expense, increased productivity, and the like). Most individuals and employers believe the hype of so-called “quick fix” products and purchase these products and services, but after using these products and services they often find them trite, even manipulative. Even more persuasive is the fact that products and services such as this result in individuals not following through and losing interest in the program altogether. Often, when they do follow through they are disappointed with—even despairing of—the results or lack thereof. They end up searching for the next quick self-improvement fix, even if they sense that they need much more.

Many people would benefit from seeking out professional services to address their lack of fulfillment or well-being, e.g., psychological and psychiatric services, medical services, other counseling or therapy, personal training, career or life coaching. But these services are often not available due to financial, geographic, social, or other constraints. These items are not always covered by insurance, and in fact pursuing these services may carry stigmas; e.g., being “crazy,” “sick” or “weak-minded.” An employer is in the best position to provide such professional services to their employees.

Employers also desire feedback. Feedback not only from the individual, but from third-party interaction with the individual. It is easy for an employee to say they went to the dentist the requisite two times a year, but without a correlating report from a dentist this data may be false. For example, if it is shown that attending the dentist twice a year reduces the risk of expensive dental procedures, a company may be interested in tracking dental visits. Other forms of tracking include third party input, objective factors such as weight, number of sick days taken etc. This allows the employer to determine the return on investment and cost savings from a capital investment.

### SUMMARY OF THE PARTICULAR EMBODIMENTS

In the present invention, systems and methods are described for an assessment tool built on a holistic well-being improvement model. The model consists of a series of assessments including, but not limited to: a well-being improvement assessment, work productivity assessment, and a clinical health risk assessment. A member would complete these assessments through an internet website, on paper, on the phone, and/or on any other medium generally known in the art. After a member participates in the assessment, the assessment is analyzed based on a series of predefined algorithms. A member is then provided with system tools and access to third-party behavior change tools and resources. Concurrently, the data is compiled for the benefit of an employer and/or provider in the form of an aggregate report in order to understand the status of the current population's well-being, and also providing the ability to predict future outcomes. The system further tracks the progress of a member on their well-being goals and behaviors.

### BRIEF DESCRIPTION OF THE DRAWINGS

Embodiments of the present invention are described in detail below with reference to the following drawings.

FIG. 1 schematically illustrates an algorithm for making an holistic well-being assessment;

FIG. 2 is an expansion of sub-algorithm 44 of FIG. 1;

FIG. 3 is an expansion of sub-algorithm 48 of FIG. 1;

FIG. 4 is an expansion of sub-algorithm 52 of FIG. 1;

FIG. 5 is an expansion of sub-algorithm 56 of FIG. 1;

FIG. 6 shows one embodiment of a recommendation algorithm;

FIG. 7 shows a screenshot of a plurality of well-being assessment dimensions in one embodiment;

FIGS. 8A-8B pictographically illustrates a LIMEADE Deployment and Usage Workflow;

FIGS. 9A-9B show screenshots of a login and registration page in one embodiment;

FIG. 10 shows a screenshot of an example well-being assessment in one embodiment;

FIG. 11 depicts a wellness education screenshot;

FIG. 12 depicts an Employee Self-Awareness assessment screenshot appearing as a consequence of engaging the Product Tour hot button of FIG. 11;

FIG. 13 depicts a “Turning awareness into action” Product Tour screenshot that appears to encourage the employee-member to set goals;

FIG. 14 shows an example screenshot of ongoing behavior change in one embodiment;

FIG. 15 shows an example screenshot of a provider dashboard in one embodiment;

FIGS. 16A-16E show a detailed employee report in one embodiment;

FIG. 17 shows a screenshot of an employer dashboard;

FIG. 18 shows a screenshot of an improve this page from the MyResults page;

FIG. 19 shows a screenshot of a MyPlan page in one embodiment;;

FIG. 20 shows a screenshot a well-being journal in one embodiment;

FIGS. 21-22 show screenshots of community forums used in one embodiment;

FIG. 23 shows an overview of the system framework;

FIGS. 24-25 show screen shots of tracker widgets.

FIGS. 26A-26D show screenshots of a member's assessment in one embodiment.

FIGS. 27-31 show screenshots of the steps an organization takes when launching the system and method.

FIGS. 32-34 show screenshots of database relationships used in one embodiment of the system.

FIG. 35 schematically illustrates a high-level System Topology of the software system that can deliver a holistic well-being assessment;

FIG. 36 illustrates functionality of invitation and interaction;

FIG. 37A illustrates a 5 point response scales within a screen view of a member survey;

FIG. 37B illustrates Assessment Results and Area View;

FIG. 38 schematically illustrates assessment Results, Area View to get to Assessment Results, Sub-Area and Sub-Area Details Views;

FIG. 39 illustrates the Create My IPlan example link leads to a set of member-specific Goal Recommendations, and the ‘Get 360° Feedback’ button leads to an invitation mechanism;

FIG. 40 illustrates peer feedback functionality and a Member Experience Flowchart;

FIG. 41 shows a screenshot of a registration page;

FIG. 42 shows a screenshot of an example assessment page;

FIG. 43 shows a screenshot of email reminder;

FIG. 44 shows a screenshot of a member's well-being;

FIG. 45 shows a screenshot of a member's results;

FIG. 46 shows a screenshot of a sample action plan;

FIG. 47 shows a screenshot of sample community forums;

FIG. 48 shows a screenshot of a sample advertisement and overview of the system and method;

FIG. 49 shows a sample email that is sent to a member to remind them of their goals and action plan;

FIG. 50 shows a screenshot of a prioritizing goals screen;

FIG. 51 shows a screenshot of a plan developed for a member;

FIG. 52 shows a screenshot of a 360° review.

### DETAILED DESCRIPTION OF THE PARTICULAR EMBODIMENTS

In general, particular embodiments include systems and methods to acquire holistic health or wellness assessments for individuals, and/or collective individuals associated with organizations. Organizations may include, but are not limited to: philanthropic entities, commercial companies, military services or governmental agencies, and service providers to multiple such organizations. Collective health may be expressed in qualitative or categorical terms, semi-quantitative, or quantitative terms.

In one embodiment of the invention, an assessment is built on a holistic Well-Being Improvement model, or ‘population vitality management’ which includes, but is not limited to: a well-being improvement assessment, a work or work well-being or productivity assessment; and a clinical health risk assessment. In one embodiment of the invention, assessments are provided by the system and method described herein. In an alternate embodiment, the assessment is provided by a third party. Additional assessments are possible, and can be readily implemented. The well-being improvement model combines disparate assessment types: it covers medical, psychological, emotional and behavior-change science; and further includes “positive psychology”; it focuses on improving well-being as the causal factor to other desirable outcomes (i.e., healthcare costs, job performance, productivity, happiness, and the like); is built for the internet, but can also perform assessments via a variety of media including, but not limited to: paper, web, phone or any other media; and/or can gather data over time.

In order to seamlessly integrate well-being, work (work well-being) and health risk assessments (40+Dimensions), a score for every area and dimension determines recommended tools/actions/resources. This is accomplished by mapping the assessment answers to dimensions and goal recommendations. A weighted score is partially based on academic analysis of the goals that help organizations drive optimal outcomes. All outcomes can be tracked by self-reported means: assessments, health-claims analysis, and organizational/financial metrics (e.g., profitability, employee job performance, employee promotions, customer satisfaction, inventory turns, etc.). The score is further based on ongoing analysis of usage of behavior change tools/resources/interventions, ratings and outcomes to determine the best outcome predictors. This also enables shorter assessments (number of questions) over time. It further includes the use of up-front assessment and ongoing assessments to improve the recommendation algorithm. Further still, this includes the use actual aggregate member behavior to develop specific recommendations. The algorithms are informed by what an individual member has done before, what behavioral tools/resources/interventions have proved successful for other participants with similar attributes, and/or what others in general have used with success. The components include, but are not limited to: Goal Popularity; Goal Tracking Tool Usage; Journal Usage; Community Forum Usage; Audio/Video Usage (e.g., podcasts, videocasts); Coaching Usage; Profile/Demographic Fields; Any available external data; Historical Chart Data; Page Use/Click Data; Feature Ratings; CSV Data (G&A, Resources, etc. by Provider/Employer); Account Settings (account type, recommendation templates, etc.); Metadata on File Types (image keywords, file uploads/downloads, etc); Email usage; Text Message Usage; and/or Phone Usage; and/or all business outcomes correlated to one or a combination of the above. Recommendations are refined over time based on any or all of the aforementioned components. Also, to progressively challenge members, the system recommends a new level (set of goals) if the simple ones are achieved.

In one embodiment of the present invention, the algorithm used to provide recommendations functionally is dynamic. Over time, the algorithm gathers additional data in order to further pinpoint the recommendations for an individual member or group of members. The algorithm begins by taking an average of all answers in a category. For example, there are five questions relating to fitness, and each question is rated on a scale from 1 to 5. The member's answers are given an average value and then ranked against averages from other categories to determine the category of greatest need. A recommendation is then provided to the member. As the member continues to add data over time, the results are weighted and based on successes and failures. For example, poor fitness may not be a result of not having the means of exercising, but may be linked to time management or eating habits. Therefore, a recommendation of a gym membership is reduced and other methods are weighted more heavily, like, for example, seeing a nutritionist. On a macro scale, the algorithm gathers data about similar people in an organization and compiles the data to determine trends. For example, one trend may include mid-level managers who are in their late thirties. In this example, they all tend to have certain exercise habits and the most successful result is providing exercise facilities in the office, because they do not attend the facility near their home. Each issue or recommendation is rated based on the original state of well-being for all individuals and based on the ongoing behavior of these individuals. The changes are tracked over time to determine the efficacy of original state predictors, and/or behavioral predictors. The result is better data over time and shorter, more targeted and effective state assessment and recommendation mechanisms.

A recommendation engine is provided that recommends a resource (aka intervention) that a Provider or Employer (or even the Employee community) wants to recommend based on reusable templates each employer and provider can fill out (and keep updated), member/employee scores and member/employee stated preferences for what they want to improve, in a simple member interface allowing for a variety of health, wellness and productivity-enhancing resources from any vendor or organization. The recommendation process (like the behavior-change process) is abstracted from any specific issue or vendor. Over time, functionality includes “platform functions,” like billing and incentive systems to track behaviors and scores across different domains outlined and different resources. Resources include, but are not limited to: well-being improvement (behavior change) resources, e.g., journal, community, content, email follow ups, goal setting and tracking, etc.; any third party resource, e.g., coaching, smoking cessation, weight loss, psychiatry, psychology, other counseling, addiction treatment, additional screenings (including biometric screenings), doctor visits, third party content, third party online tools (even if similar to or overlapping with ours), and the like; and/or ongoing tracking of individual goals tied to well-being improvement (behavior change) using emails, gadgets/widgets/portlets, phone/text message, and the like. In one embodiment of the present invention, a member can get a plan that maps to his desires, his assessment scores (in many areas), what his employer wants him to work on, and what his provider wants him to work on.

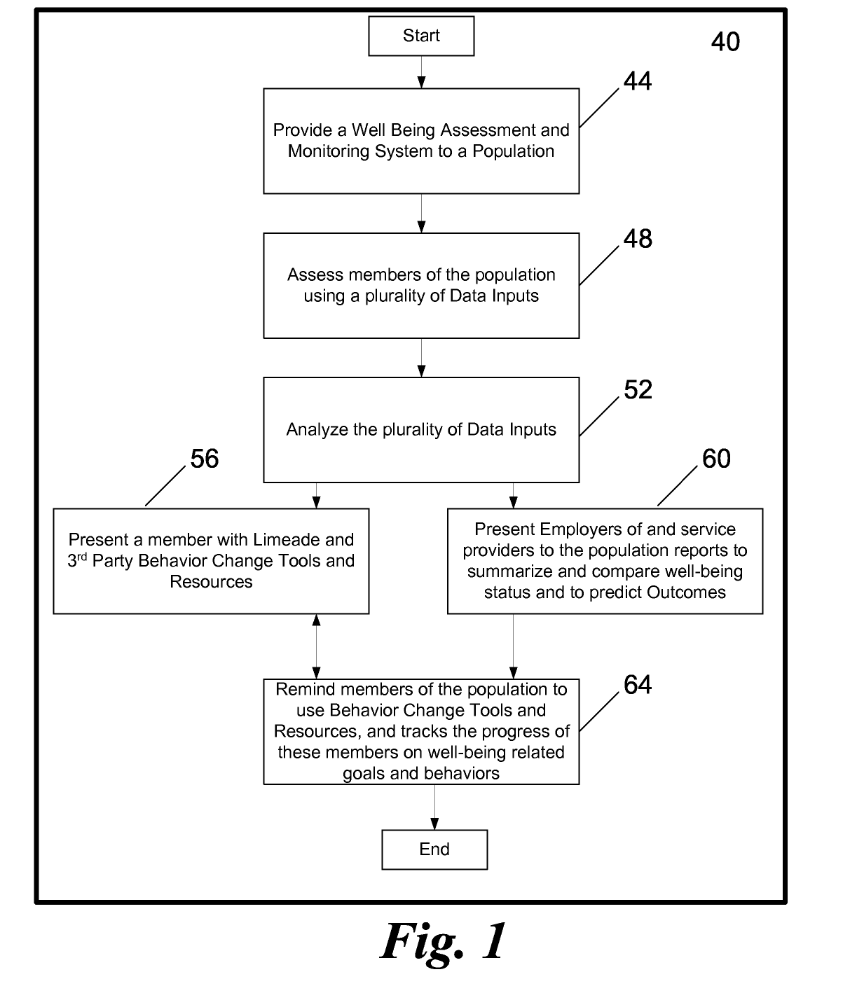

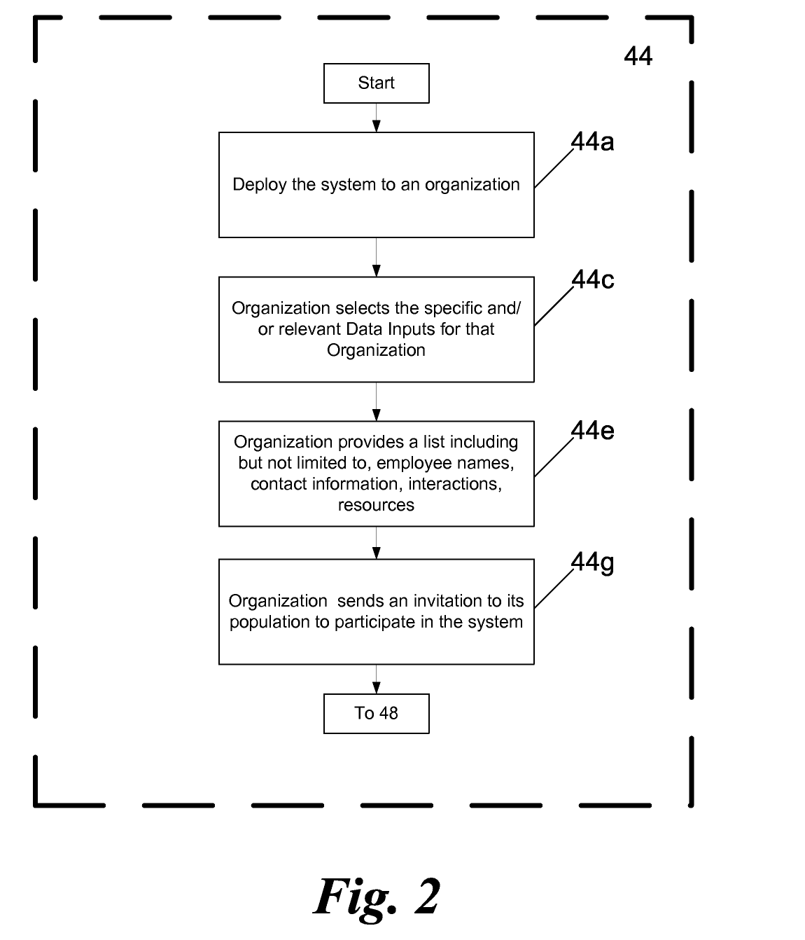

FIG. 1 schematically illustrates an algorithm 40 for making a holistic well-being assessment. At block 40 a well being assessment and monitoring system is provided to a population. In one embodiment the assessment and monitoring system is provided to a provider and/or an employer. The provider and/or the employer determine the specific and relevant assessments and provide the population with a notification that the system is in place. This process is further described in FIG. 2.

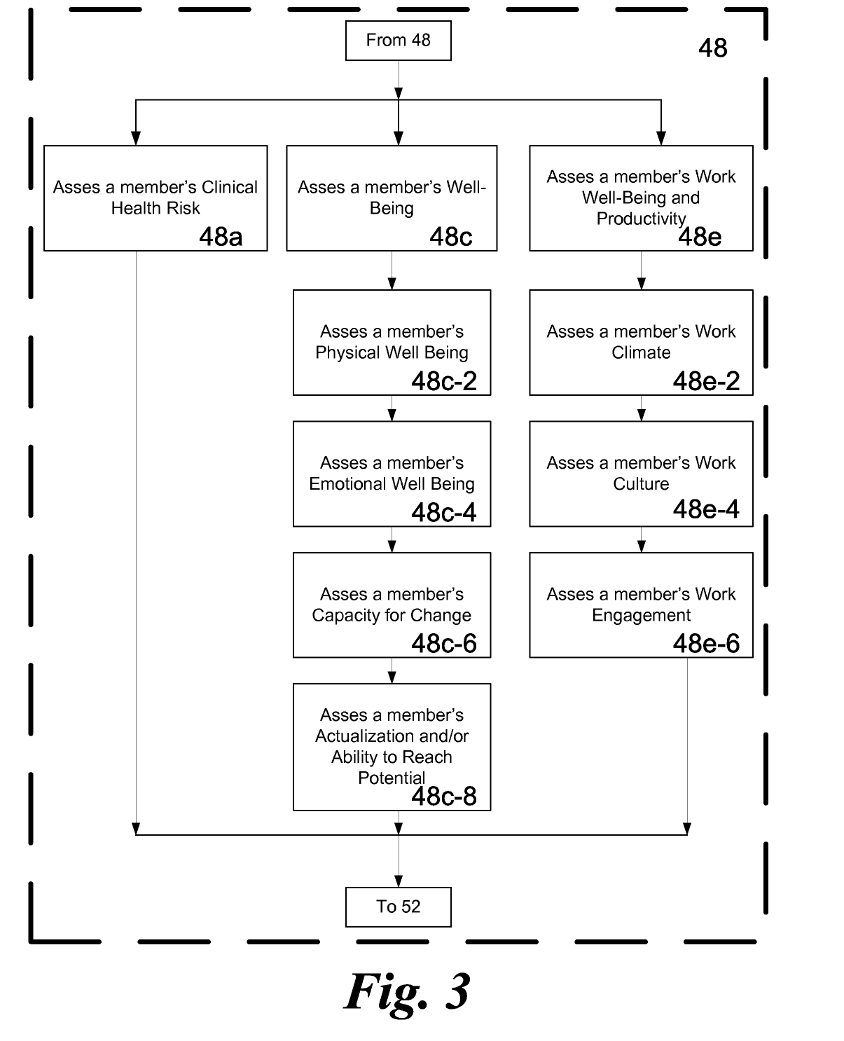

At block 48, a member of a population is assessed using a plurality of data inputs. The inputs include assessments in a number of areas that make up a member's overall well-being. These assessments may consist of assessments given through the software or may consist of third party assessments. This process is further described in FIG. 3.

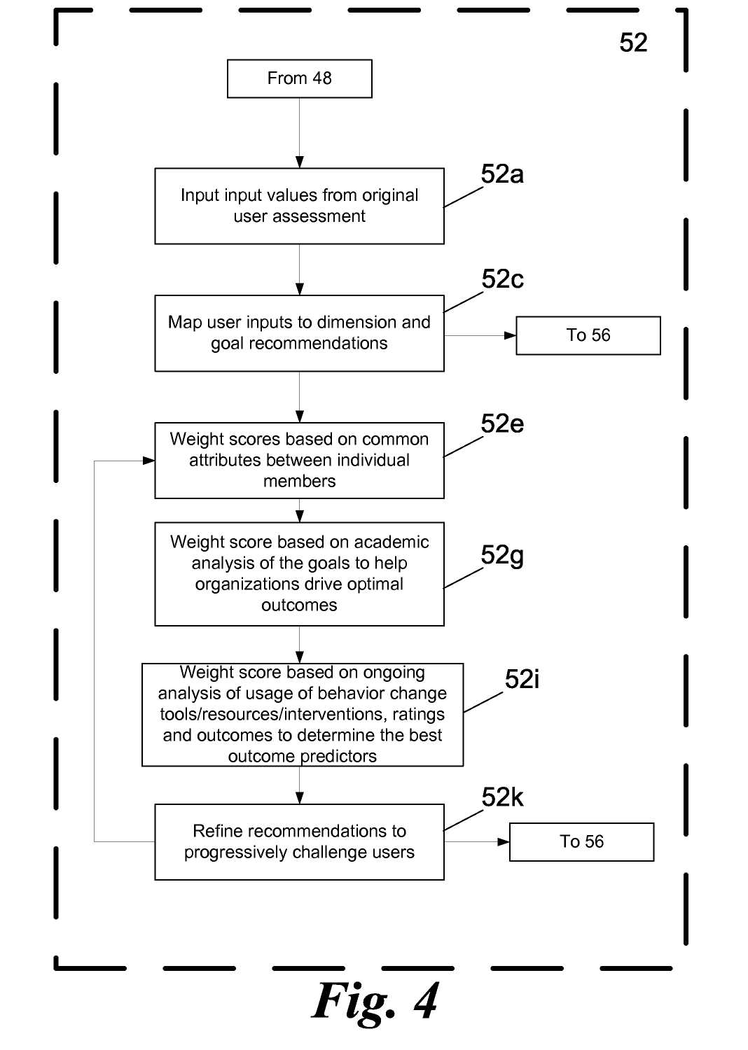

At block 52, the plurality of data inputs are analyzed. The software based assessments and the third party assessments are analyzed mathematically in order to determine which third party change tools and resources (block 56) provides an aggregate roll-up of members' quantitative results (see block 60). This process is further described in FIG. 4.

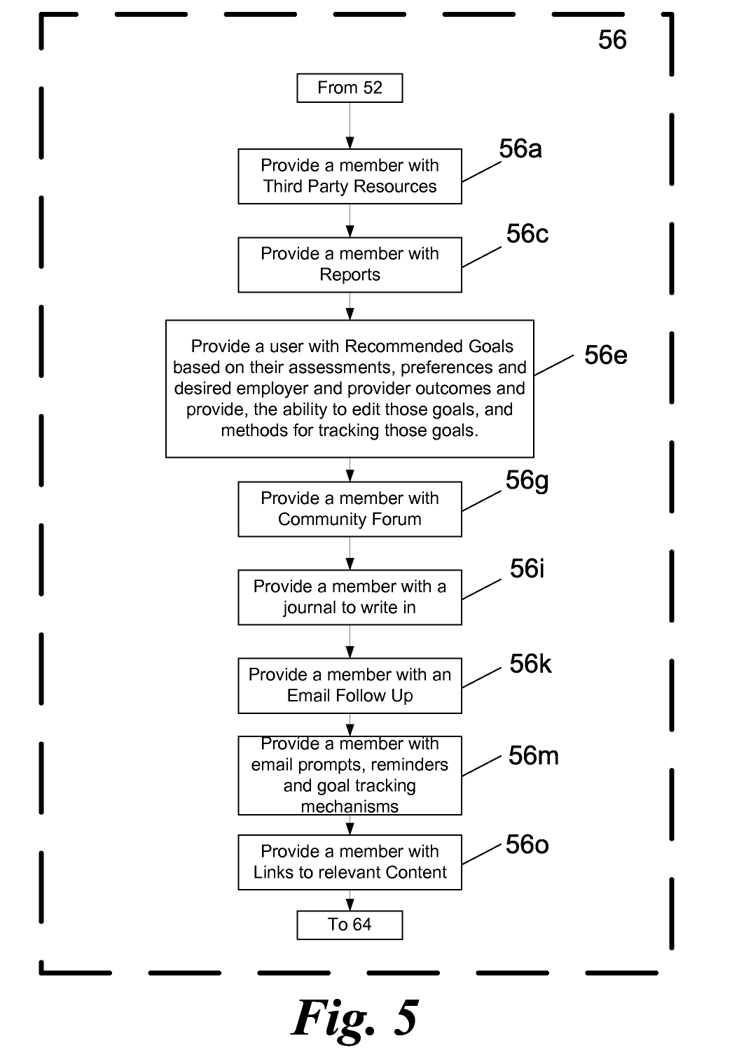

At block 56, a member is presented with Limeade and third party behavior change tools and resources. This process is further described in FIG. 5.

At block 60, an employer of and service providers to the population are provided with reports to summarize and compare well-being status and predict outcomes. The employer and/or provider is not shown the members' individual responses, but the system method takes a mathematical calculation and provides the employer and/or provider with a quantitative result. For example a question for a member may be: do you smoke, and an employer and/or provider would not see each individual answer but would see a percentage of people who do smoke at that organization. This allows for a corrective action to be determined. It further categorizes the answers and gives an average score for each category; these categories include but are not limited to managing stress and anxiety; autonomy; life meaning; resilience and/or self efficacy. The employer and/or provider can then compare those to an average and determine where their organization stands in comparison. These reports give the employer and/or provider real time quantitative results that allow them to monitor the well-being of the organization at that moment in time.

At block 64, members of the population are reminded to use behavior change tools and resources. Further, the progress of the members is tracked and based on well-being related goals and behaviors. The system using multiple media, contacts the member to remind them to take the assessments; update results; write it journals or microjournals related to those goals, and achieve their goals. It further includes an incentive based program. The program tracks the progress of the members through self reporting and reporting by third parties. As an example a third party may report how often the member attends counseling, where a member would self report levels of stress, productivity etc. Objective measures may be used such as, weight and/or blood pressure. These inputs are received and tracked for improvement and incentive purposes. As new information is input into the system, the behavior change tools and resources (see block 56) are adapted to correlate with the updated results.

FIG. 2 is an expansion of sub-algorithm 44 of FIG. 1. At block 44a, the system is deployed to an organization. This is comprised of providing branding and/or relevant assessments. At block 44c the organization selects the specific and/or relevant data inputs for that organization. An organization may have a focused assessment that they are interested in and therefore may limit the scope of the assessments. At block 44e, the organization provides a list including but not limited to, employee names, contact information, interactions and/or resources. These data inputs lead to more specific data inputs for the organization. At block 44g the organization sends an invitation to its population to participate in the system.

FIG. 3 is an expansion of sub-algorithm 48 of FIG. 1. In one embodiment, there are three overarching assessment themes; clinical health risk, well-being and work well-being and productivity. A member may be assessed on each of these, none of these or some combination of the three. In alternate embodiments other assessments are used. At block 48a a member is assessed on their clinical health risk. Clinical health risk includes items such as physical health conditions, blood tests, diseases, propensity for illness, and/or health histories. At block 48c a member's well-being is assessed. In assessing the well-being of a member the system and method assesses at block 48c-2, a member's physical well-being; at block 48c-4, a member's emotional well being; and at block 48c-6, a member's capacity for change is assessed. At block 48c-8, a member's actualization and/or ability to reach potential is assessed. At block 48e a member's work well-being and productivity is assessed. In assessing a member's work well-being and productivity, at block 48e-2, a member's work climate is assessed, at block 48e-4, a member's work culture is assessed and at block 48e-6, a member's work engagement is assessed. These assessments are based on a numerical scale and are stored numerically by question and then categorized.

FIG. 4 is an expansion of sub-algorithm 52 of FIG. 1. At block 52a numerical values are input from the original member's assessment. The values are imported and are mapped to dimensions and goal recommendations at block 52c. This completes the initial assessment and the results are output to block 56 in FIG. 1. As the member continues to participate and other members in the organization provide responses and participate; at block 52e the scores are weighted based on common attributes between the individual members. Common attributes include those with common age, position, sex, race etc. The system can weigh more heavily those questions and those recommendations that had the desired effect on similarly situated individuals. At block 32g a score is weighted based on academic analysis of goals that help the organization drive optimal outcomes. The system optimizes results to not only help the member, but to improve the member's work productivity. Those questions and recommendations that are more likely to result in an increased productivity are given a higher weight value and over time are adapted to optimize results. At block 52i a score is weighted based on ongoing analysis of usage of behavior change tools/resources/interventions, ratings and outcomes to determine the best outcome predictors. As the member participates in the system tracking occurs. As the system tracks results weight values are adjusted to weight those recommendations that lead to positive results. At block 52k, recommendations are refined to progressively challenge members. Goals and recommendations are changed and adapted to keep them challenged and engaged.

FIG. 5 is an expansion of sub-algorithm 56 of FIG. 1. At block 56a, a member is provided with third party resources. These resources range from in-person or telephonic counseling, to health care visits, to fitness based coaching, to smoking cessation, to weight loss and more. At block 56c, a member is provided with reports. These reports include analysis of well being, overall results and show recommendations for the member. At block 56e a member is provided with recommended goals based on their assessments, preferences and desired employer and/or provider outcomes and provides a member the ability to edit those goals and methods to track those goals. The goals are fluid and are changed as shown in FIG. 4. For example a goal may include productivity benchmarks, weight loss calculations or any other quantifiable goal. At block 56g a member is provided with a community forum. A community forum is used to discuss how the program is working for the individual, share experiences and to post victories. The forum serves as a support system and a motivator and encourages all members to accomplish goals and to help others complete their own goals. At block 56i a member is provided with a journal. The journal is used to track individual results and provides an assessment tool for continued progress. At block 56k, a member is provided with an email follow up to remind them to continue to strive to reach the goals and recommendations that have been provided. At block 56m a member is provided with further email prompts, and goal tracking mechanisms to further remind the member to participate. At block 56o the member is provided with links to relevant content. This content includes websites, literary works, podcasts and other media distribution systems in order to provide the member more information on how to improve their well-being.

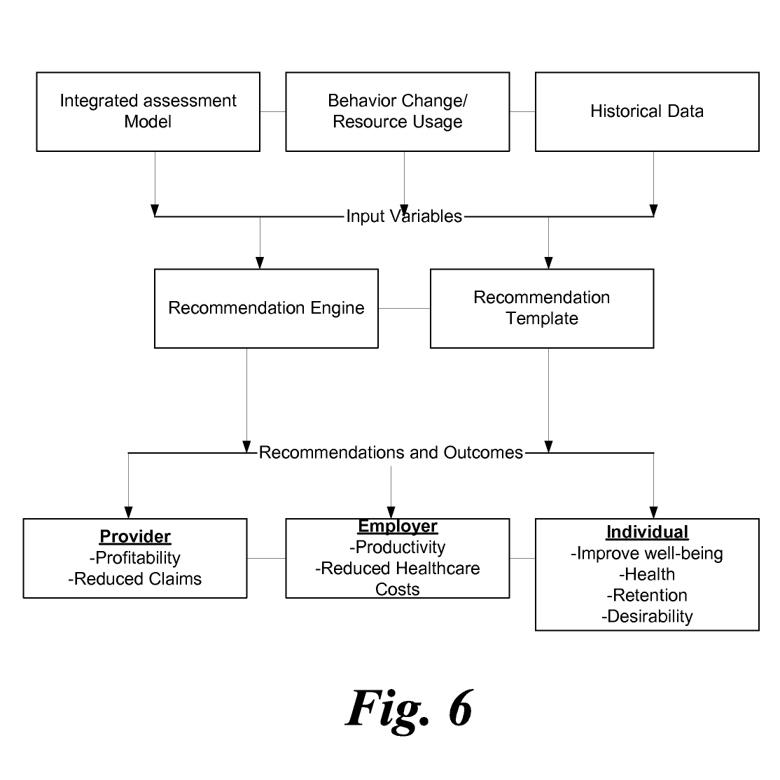

FIG. 6 shows one embodiment of a recommendation algorithm.

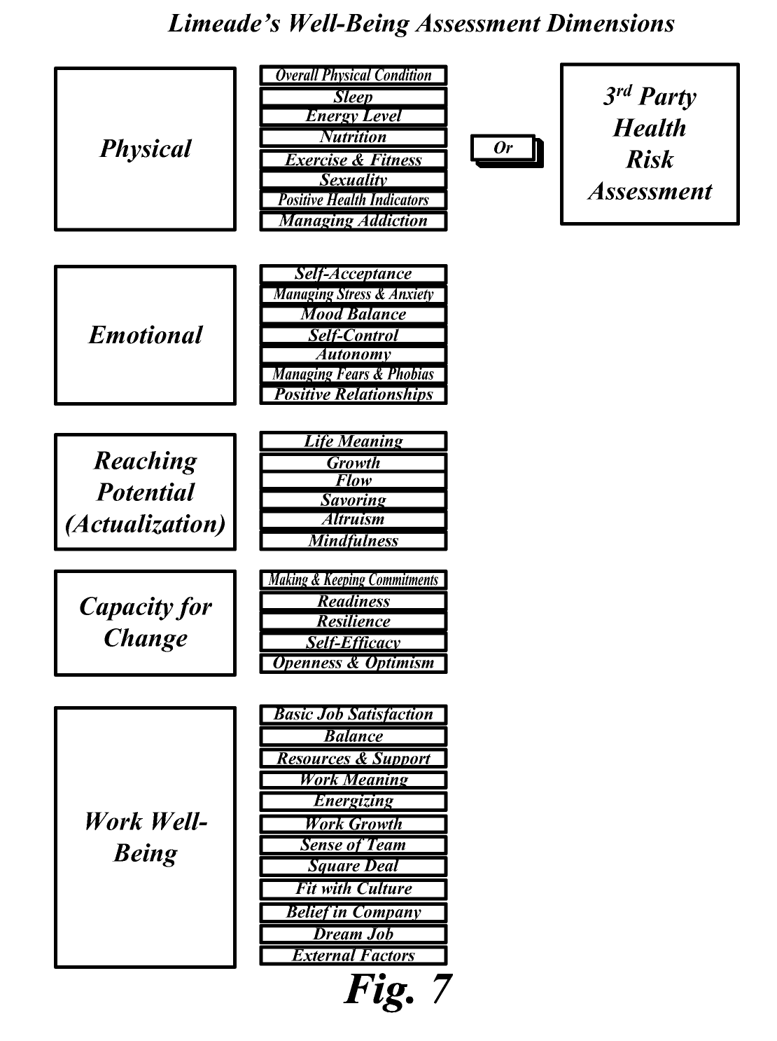

FIG. 7 shows a screenshot of a plurality of well-being assessment dimensions in one embodiment. A member takes an assessment in five parts. The parts, called “life sub-areas” can be taken one at a time or all at once benefit providers and employers can decide which life sub-areas to offer. Each Life Area has several dimensions, and each dimension is causally linked to one or more of the following business outcomes for employers: Performance/Productivity, Absenteeism, Claims/Injuries/Accidents, Health Care Costs, Job Satisfaction, Well-Being, Overall Health Attrition/Intention to Leave. The components as shown in the Area List include: Life Areas that are organized into sub-areas having easy-to-understand concepts that permit and encourage people to self-assess at any point in time. The sub-areas fit into Areas as described in sub-area List. Sub-areas are concepts hypothesized and statistically proven in published research to correlate to overall well-being, organized into sub-areas that are easy for average people to understand.

Physical is an Area that contains the sub-sub-areas overall physical condition, sleep, energy level, nutrition, exercise & fitness, sexuality, positive health indicators, and managing addiction. Overall physical condition is a sub-area that contains questions about sickness's and weight. Sleep is a sub-area that contains questions about sleep habits and sleep quality. Energy level is a sub-area that contains questions about energy level during work, overall energy levels etc. Nutrition is a sub-area that contains assessment questions about, e.g. nutrition-related topics like quality and quantity of food consumed. Exercise and Fitness is a sub-area that contains assessment questions about, e.g. physical activity and inactivity. Sexuality is a sub-area that contains assessment questions about, e.g. emotional and physical satisfaction with sex life, frequency of sexual activity, role of sex in life overall. Positive Health Indicators is an Area that contains assessment questions about laughter, stress etc. Managing addiction contains questions about when you drink, what triggers an addiction etc.

Emotional is an Area that contains the Sub-sub-areas Self-Acceptance, Managing Stress & Anxiety, Mood Control, Self-Control, Autonomy, Managing Fears & Phobias, and Positive Relationships. Self-Acceptance is a sub-area that contains assessment questions about, e.g. the member's sense of his/her own dignity and worth, and self-trust. Managing Stress & Anxiety is a sub-area that contains assessment questions about, e.g. fear, apprehension, worry, obsessive behaviors, and physical sensations such as nausea, chest pain, and shortness of breath. Mood Control is a sub-area that contains assessment questions about, e.g. depression and Affect Balance. Self-Control is a sub-area that contains assessment questions about, e.g. control of one's emotions and understanding how they affect Well-being. Autonomy is a sub-area that contains assessment questions about, e.g. the ability to self-regulate, resist societal pressure, evaluate by personal standards. Fears & Phobias is a sub-area that contains assessment questions about, e.g. strong, persistent, unreasonable or out-of-control fears. Positive Relationships is a sub-area that contains assessment questions about, e.g. love, fulfillment, companionship, and general positive relations with others.

Actualization is a sub-area that contains Sub-sub-areas Life Meaning, Growth, Flow, Savoring, Altruism, and Mindfulness. Life Meaning is a sub-area that contains assessment questions about, e.g. purpose, spirituality, values, self-worth, and work/career. Growth is a sub-area that contains assessment questions about, e.g. a feeling of continued personal development. Flow is a sub-area that contains assessment questions about, e.g. the pursuit of optimal experiences and control over one's inner life. Savoring is a sub-area that contains assessment questions about, e.g. awareness of and attention to pleasure, gratitude, kindness, and generosity. Altruism is a sub-area that contains assessment questions about, e.g. giving to and helping others. Mindfulness is a sub-area that contains assessment questions about, e.g. awareness and acceptance of self and surroundings, competence in managing the environment and choosing situations that match your personal needs.

Capacity for Change is a sub-area that contains the Sub-sub-areas Making & Keeping Commitments, Resilience, Readiness, Self-Efficacy, and Openness & Optimism. Making & Keeping Commitments is a sub-area that contains assessment questions about, e.g. the ability to truly commit, document commitment, and follow through and sustain change. Resilience is a sub-area that contains assessment questions about, e.g. the ability to bounce back. Readiness is a sub-area that contains assessment questions about, e.g. desire, importance, and time allotted and support for change. Self-Efficacy is a sub-area that contains assessment questions about, e.g. beliefs about capabilities to perform in ways that affect well-being. Openness & Optimism is a sub-area that contains assessment questions about, e.g. attitudes and habits of thinking.

Work Well Being is a sub-area that contains the Sub-sub-areas Basic Job Satisfaction, Balance, Resources & Support, Work Meaning, Energizing, Work Growth, Sense of Team, Square Deal, Fit with Culture, Belief in Company, Dream Job, and External Factors. Job Satisfaction is a sub-area that contains assessment questions about, e.g. skills and abilities fit well with the job. Balance is a sub-area that contains assessment questions about, e.g. working a reasonable number of hours. Resources and Support is a sub-area that contains assessment questions about, e.g. having the resources to be able to do the job. Work Meaning is a sub-area that contains assessment questions about, e.g. contributing something at work that matters. Energizing is a sub-area that contains assessment questions about, e.g. feeling valued by the employer. Work Growth is a sub-area that contains assessment questions about, e.g. upward mobility in the organization. Sense of Team is a sub-area that contains assessment questions about, e.g. trust in co-workers. Square Deal is a sub-area that contains assessment questions about, e.g. paid fairly for the work that is done. Fit with Culture is a sub-area that contains assessment questions about, e.g. the companies values are in line with personal values. Belief in Company is a sub-area that contains assessment questions about, e.g. understanding and believing in the future direction of the company. External Factors is a sub-area that contains assessment questions about, e.g. happiness with the location of residence.

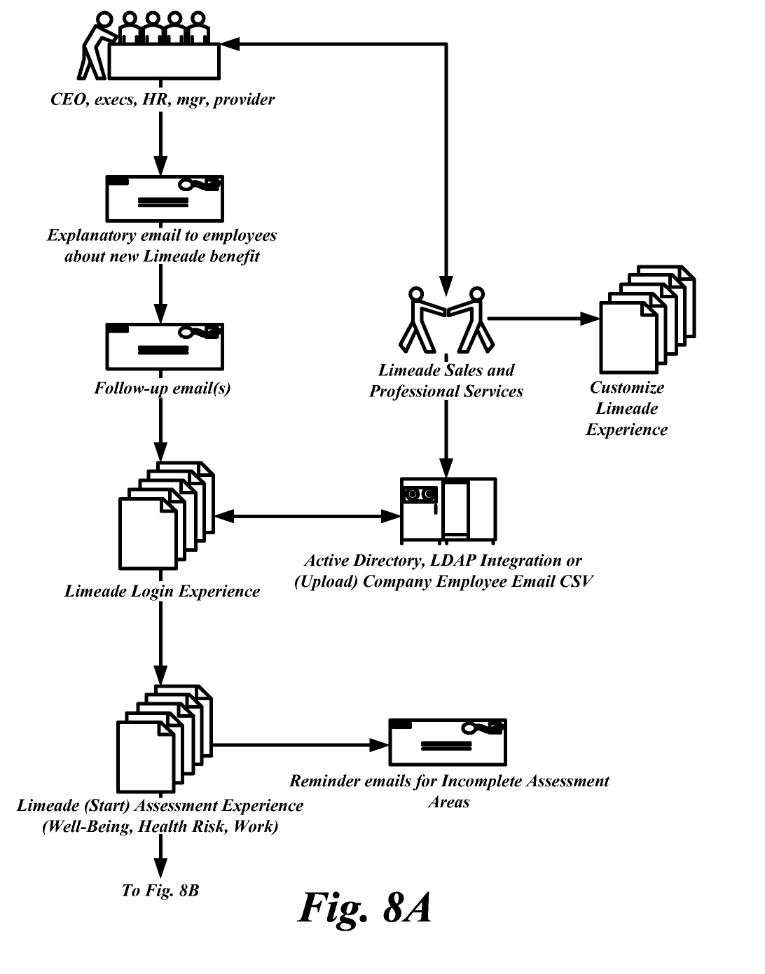

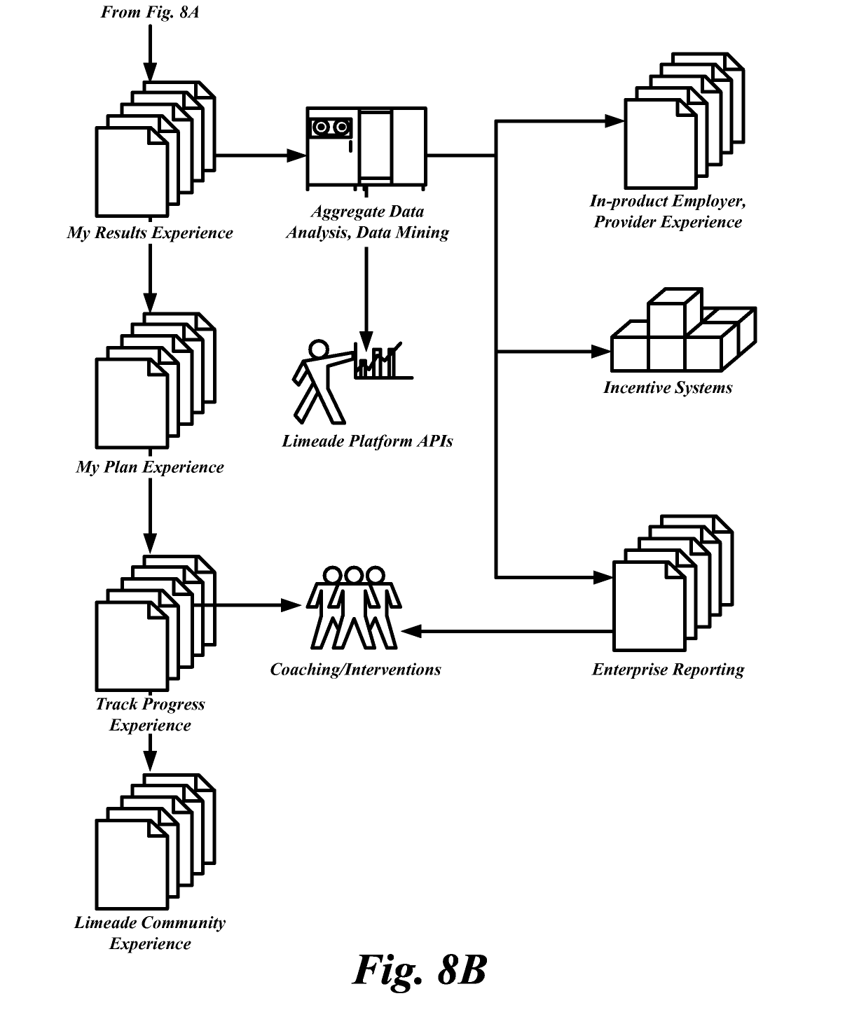

FIGS. 8A-8D pictographically illustrate a LIMEADE Deployment and Usage Workflow at high level to determine the health status in wellness terms of an individual, an organization of individuals, or multiple organizations of individuals. Organizational leaders, for example, CEOs, HR, company mangers, or benefit providers interact with the LIMEADE sales and professional services who customize the Limeade Experience with internet accessible or other computer readable media to develop interactive interfaces for determining the wellness of individuals and the productivity drivers impacting the organization associated with the wellness level of the organization's employees. The Limeade Experience is promoted by the organization leaders to its employees via explanatory emails or other communications conveyed by alternate media, for example video, DVDs, radio, or regular mail. Follow-up communications by email or alternate media are made to encourage employee recruitment to participate in wellness assessment. Committed employee-members then interact with the Limeade interface screenshots and are stored in an active directory capable of lightweight directory access protocol (LDAP) integration to upload the organization's or company employee email in a comma separated value (CSV) format for subsequent searching, grouping, and accessing. Thereafter, the well-being, health risk, and work related assessments may be undertaken with follow-up communication reminders by electronic or alternate media for those assessment areas deemed incomplete.

The assessments are then analyzed and formatted for the individual employee in a series of screenshots including the My Results Experience, My Plan Experience, Track Progress Experience, and the Limeade Community Experience. The My Results Experience of multiple employees is subjected to aggregate data analysis and data mining for eventual dashboard reports of wellness assessment of participating employees to the organization and/or multiple organizations, along with reports to benefit providers via a Limeade Platform application programming interface (API). From the data mining and aggregate data analysis, a Benefits provider Experience and an In-product Employer Experience is obtained. Incentive systems may also be developed to provide productivity drivers for employers to encourage wellness improvement of their employees to reduce healthcare costs by positive action behavior modification. The incentive systems may be incorporated in an enterprise reporting interface that may be utilized by coaching and other interventions as part of the Track Progress Experience.

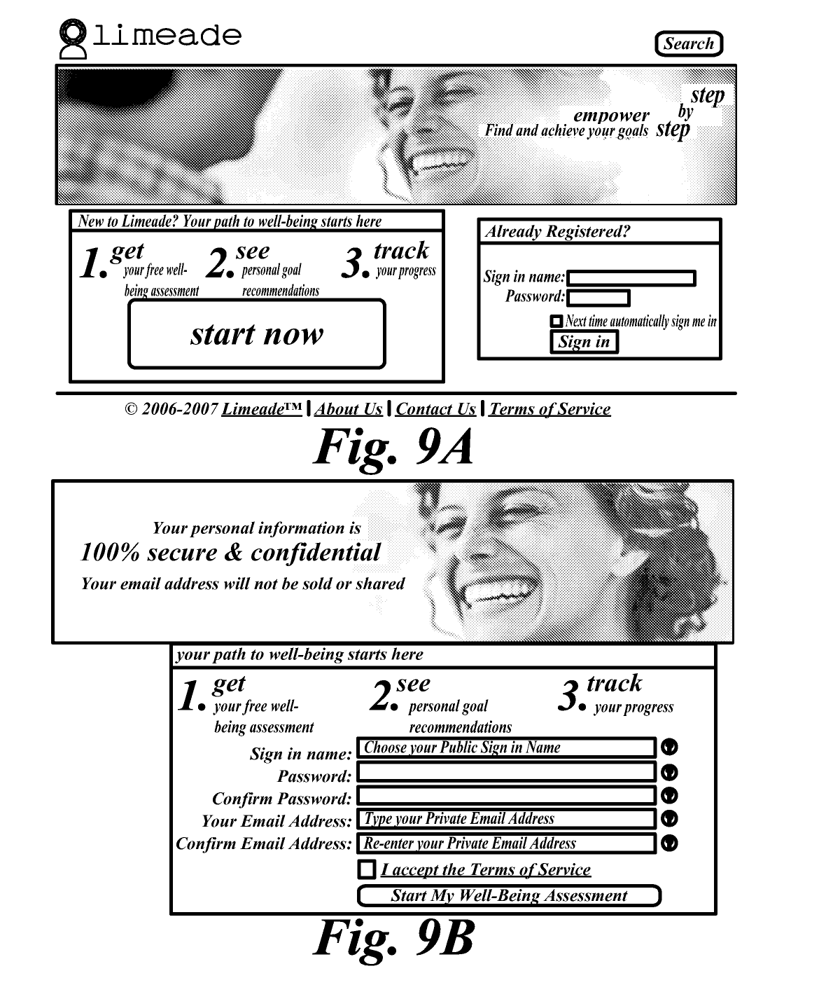

FIGS. 9A-9B show screenshots of a login and registration page in one embodiment.

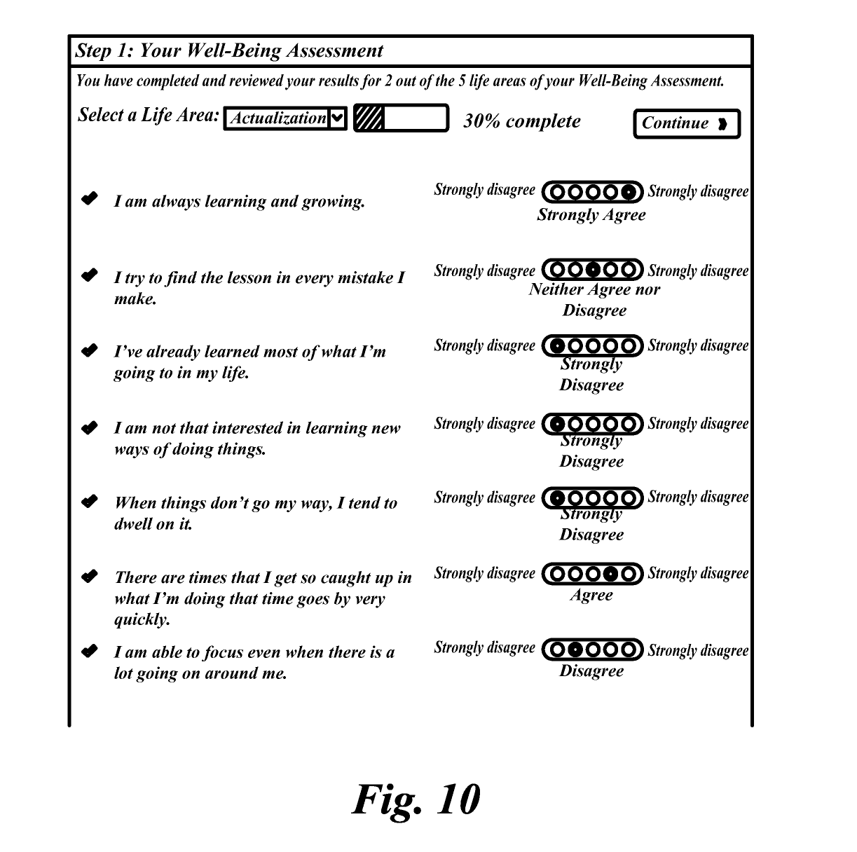

FIG. 10 shows a screenshot of an example well-being assessment in one embodiment.

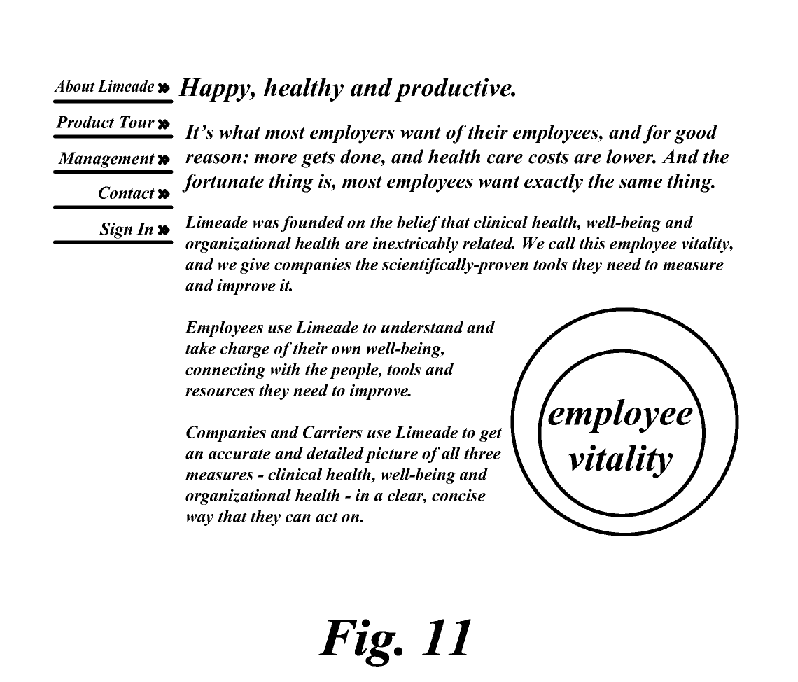

FIG. 11 depicts a wellness education screenshot in terms of “About Limeade”, the wellness tools offered to Limeade assessment participants, and a snapshot of the association of employee wellness to the operations and wellness of the participant's organization or employer. A side panel of button-engageable to bring fourth new screenshots include “About Limeade”, “Product Tour”, “Management”, “Contact”, and “Sign in”.

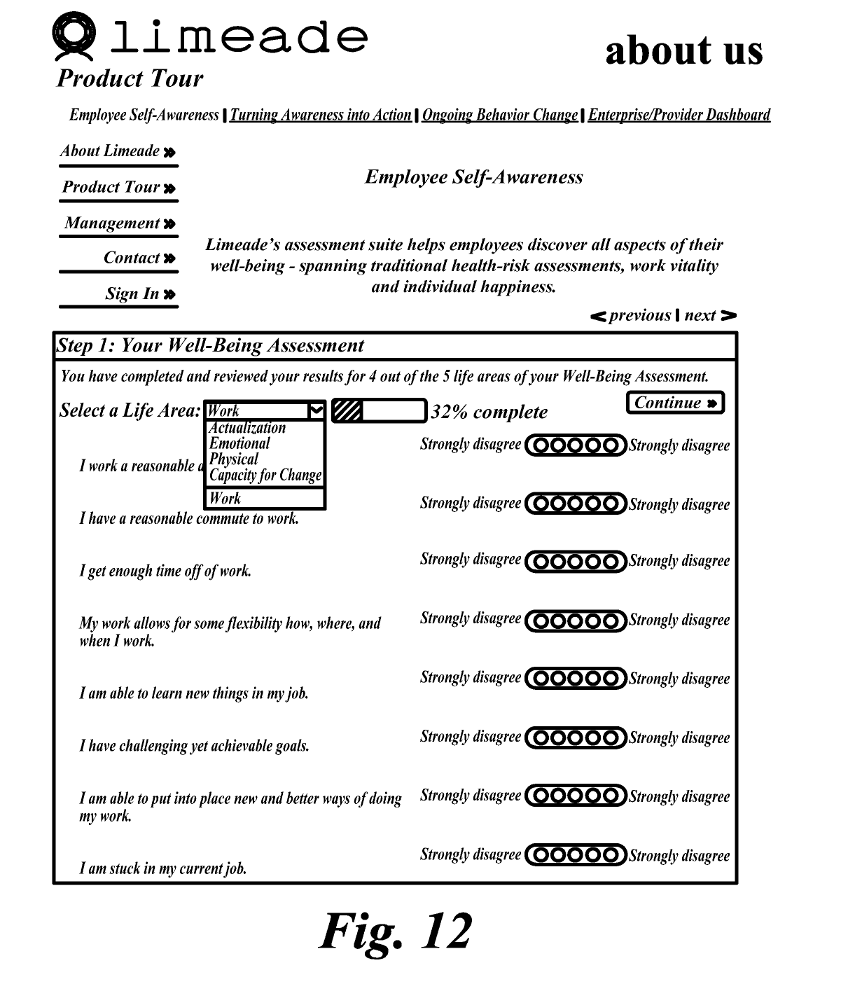

FIG. 12 depicts an Employee Self-Awareness assessment screenshot appearing as a consequence of engaging the Product Tour hot button of FIG. 11. The screenshot includes a questionnaire utilizing a numerically ranked answer 5 point incremental scale ranging between strongly disagree and strongly agree for subject matters selectable by pull down menus. The subject matters include work, actualization, emotional, physical, and capacity for change. A horizontal bar is shown for that proportion of the questionnaire survey completed—in this example 32%. Here the questionnaire includes questions covering job satisfaction, job freedom, and whether goals are satisfactory or challenging enough to the member.

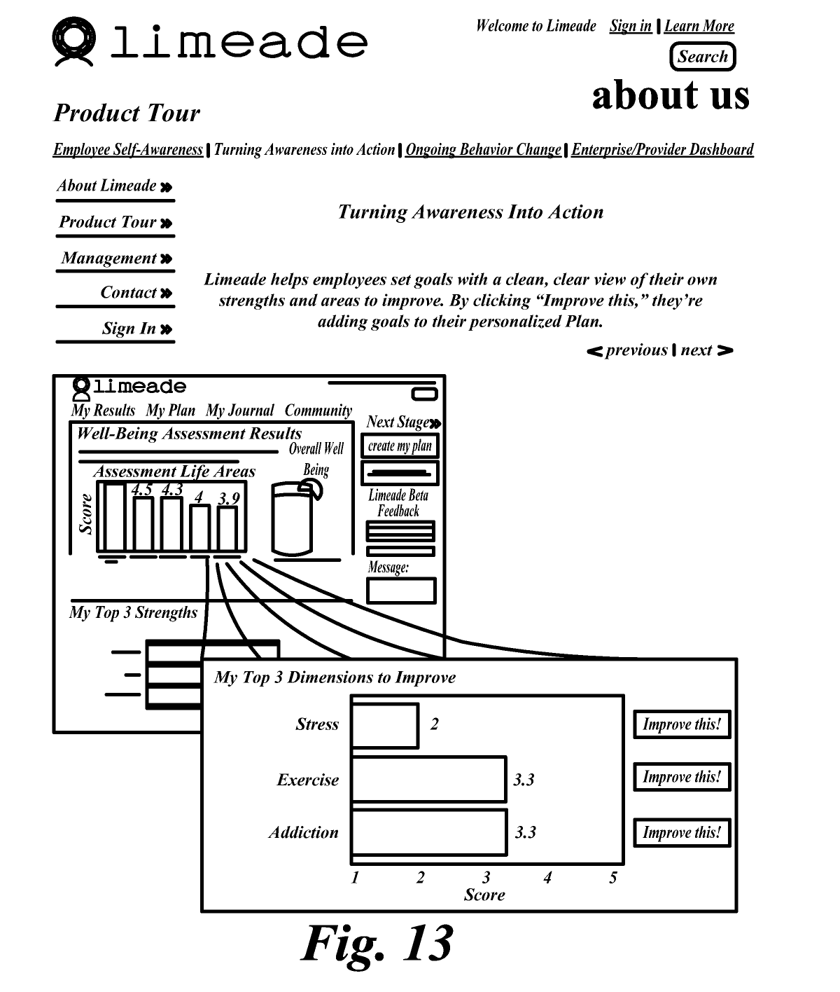

FIG. 13 depicts a “Turning awareness into action” Product Tour screenshot that appears to encourage the employee-member to set goals. The goals depicted here as part of a graphic “Dimensions to Improve” includes improving stress, improving exercise, and reducing to eliminating addiction.

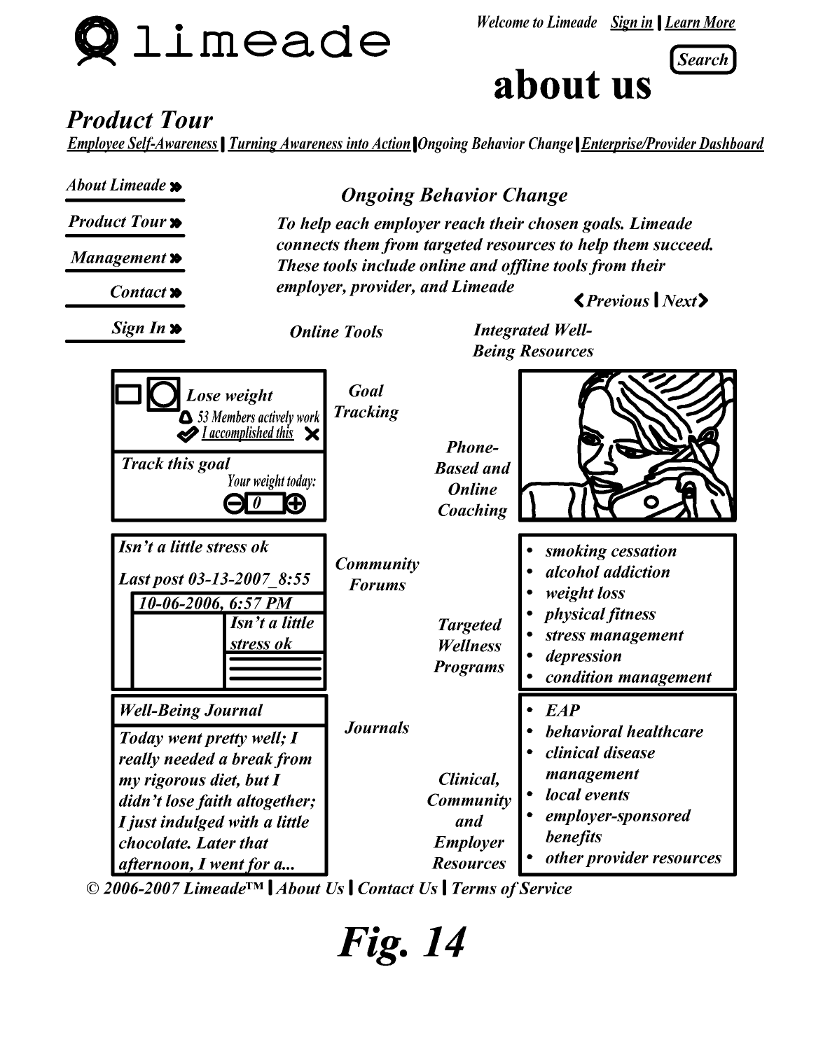

FIG. 14 shows an example screenshot of ongoing behavior change in one embodiment. The screenshot shows the connection between online tools and resources to help a member succeed.

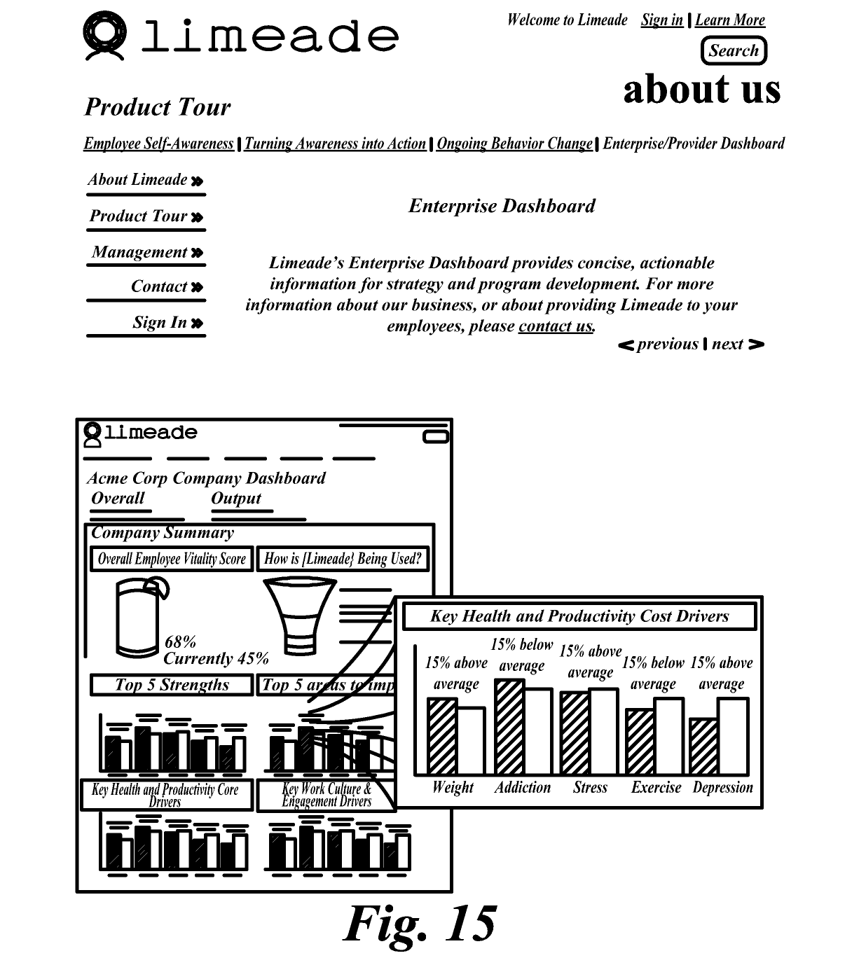

FIG. 15 shows an example screenshot of a provider dashboard in one embodiment.

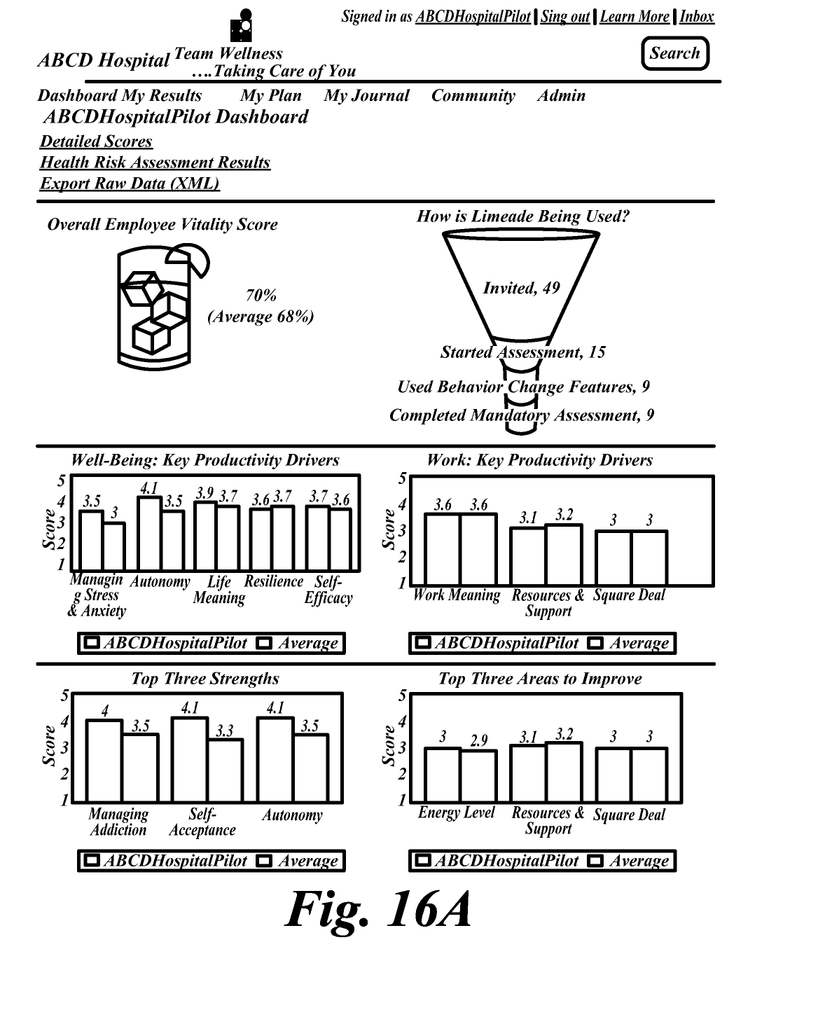

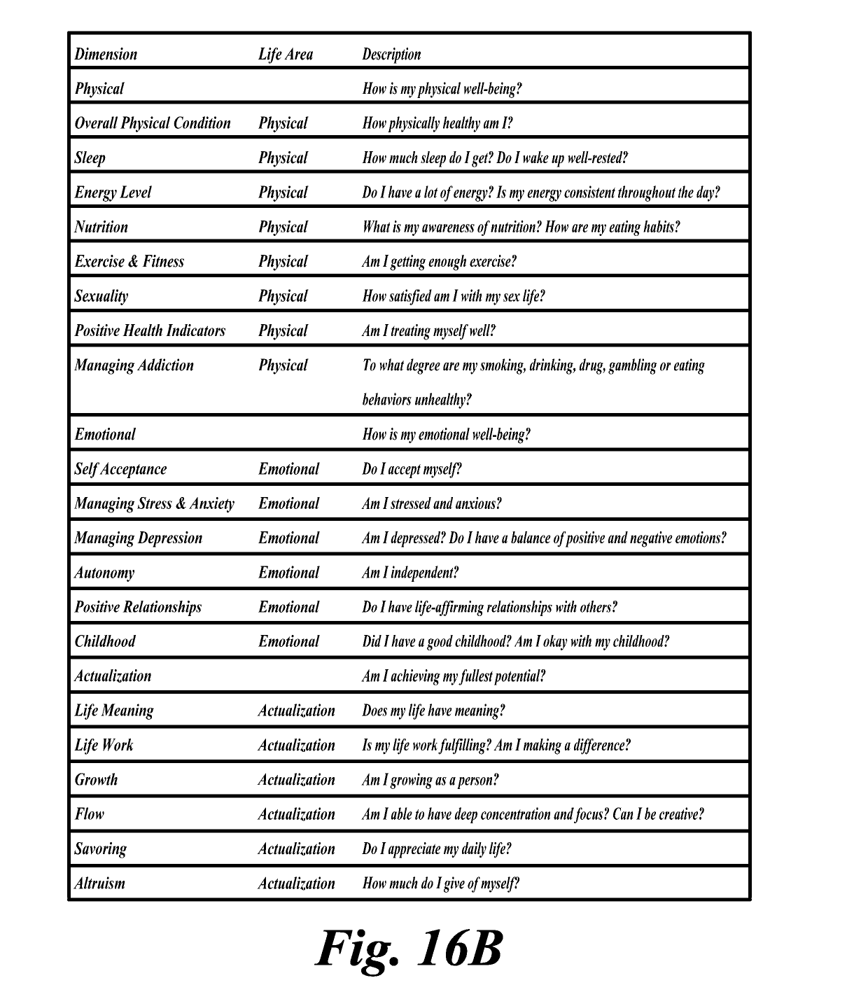

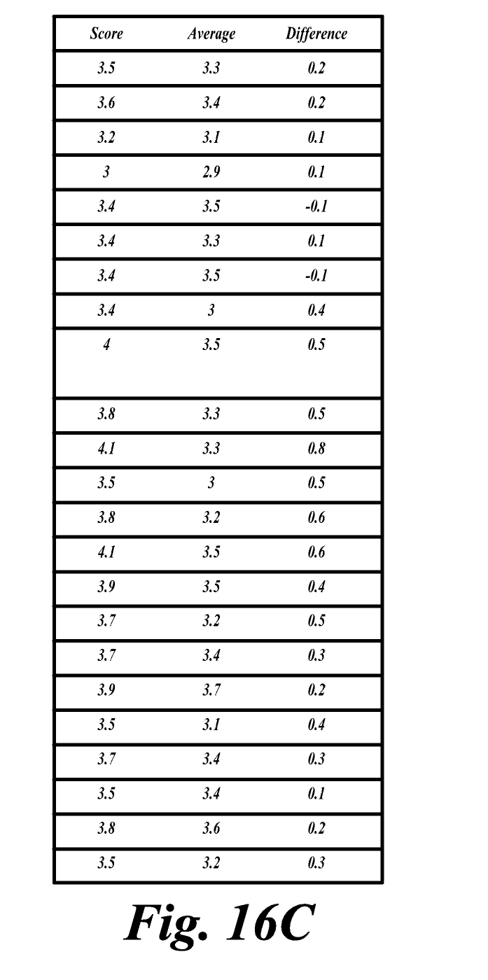

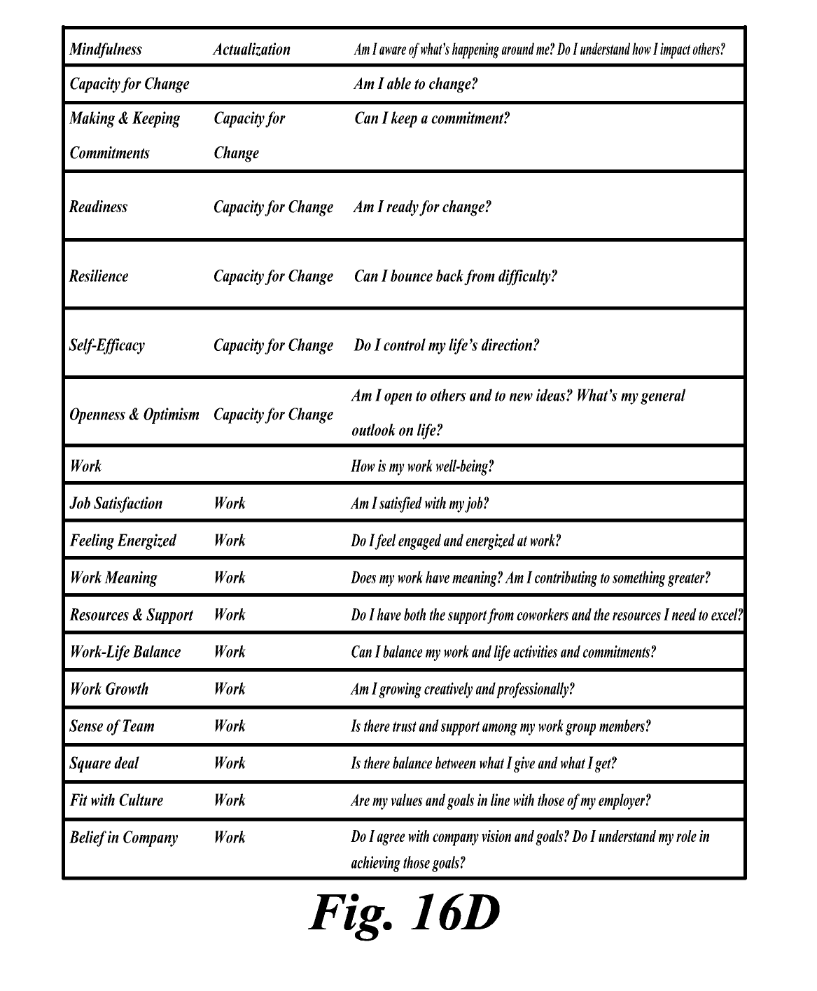

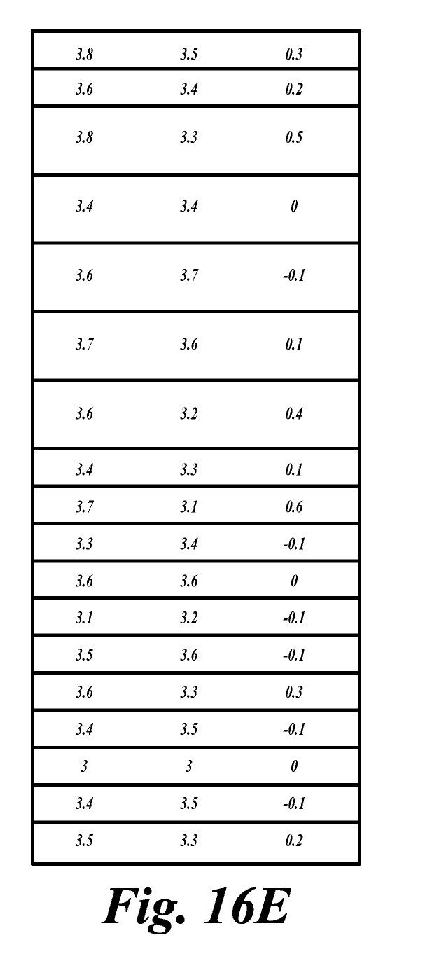

FIGS. 16A-16E show a detailed employee report in one embodiment. When members are done with their assessments, at least three types of reports are generated, individual end-member or employee reports, dashboard reports that aggregate reports for employers, and dashboard reports that aggregate data for benefit providers. Here is an employee report called MyResults, which includes summaries of the Life Areas (including Clinical health Risk assessments, not shown here), an overall score, top strengths, top dimensions to improve, detailed life area and dimension scores, chart details that popup to give descriptions of that dimension, the member's score, resources for learning more (e.g., articles and books), forums to discuss the topic, podcasts on the topic, and the opportunity to click “Improve This”, which adds a goal related to the dimension to the member's MyPlan page—their personal improvement plan.

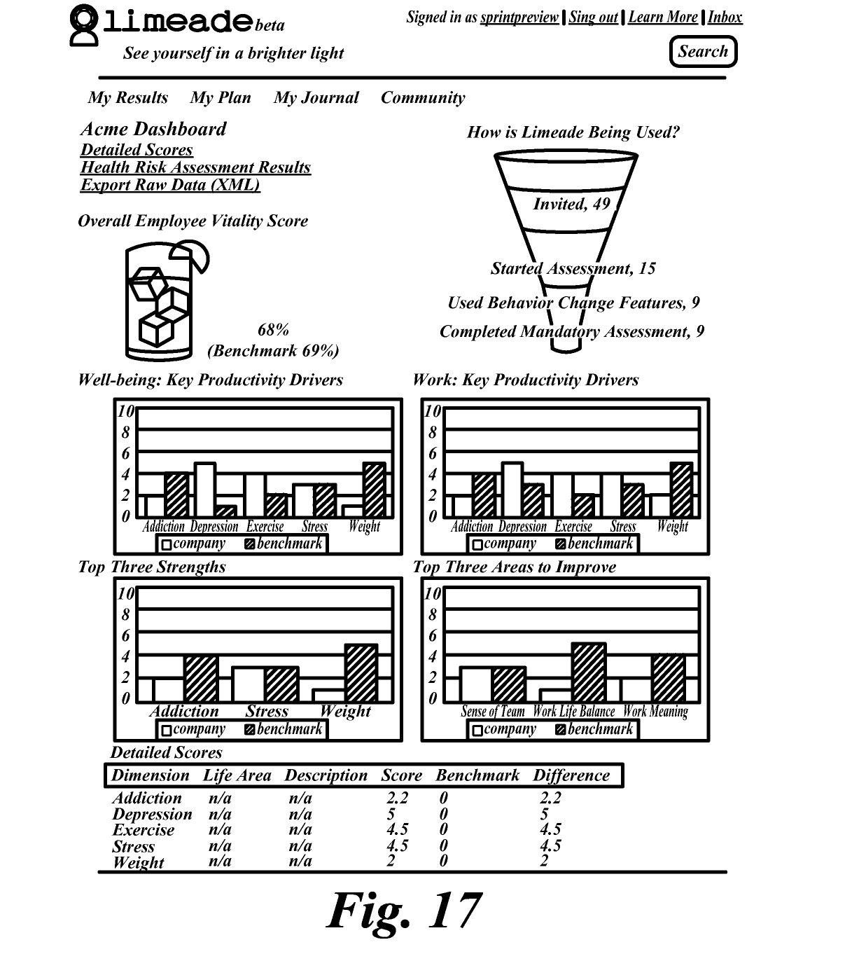

FIG. 17 shows a screenshot of an employer dashboard. In addition to individual end member reports, employers and providers receive dashboard report summaries that aggregate and filter individual data. The Provider and Employer Dashboards are similar but not the same. The Provider Dashboard aggregates data across multiple employers. Underlying each dashboard is a database of aggregated ‘Population Vitality’ information.

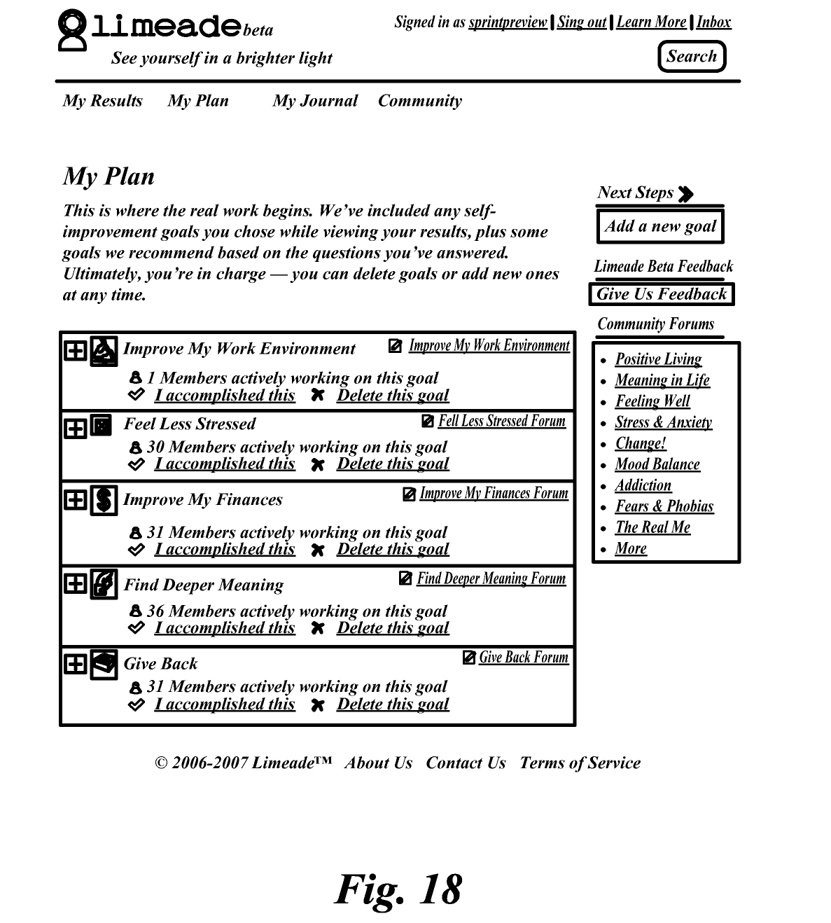

FIG. 18 shows a screenshot of an improve this page from the MyResults page. This screen shows tailored recommendations for the member based on their assessment results and preferences.

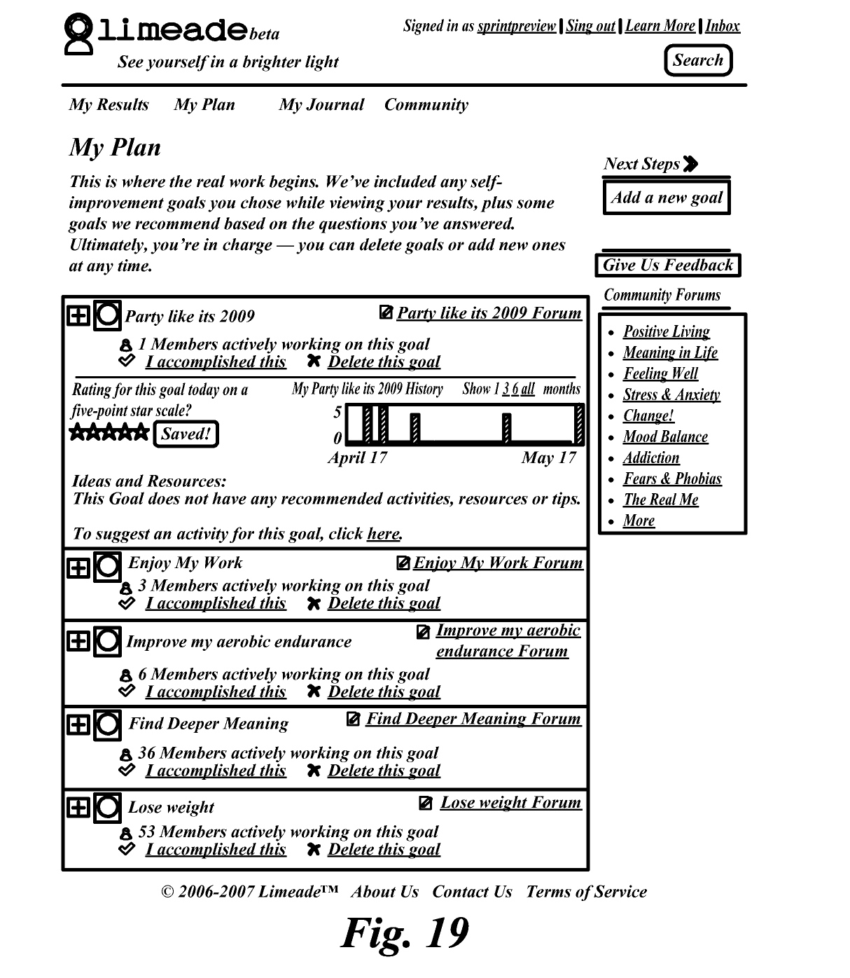

FIG. 19 shows a screenshot of a MyPlan page in one embodiment. Each MyPlan page has a place to track progress toward goals using various measurement tools, including journals, numerical scores, weekly numerical scores, and 5-point visual scales (e.g., smiley faces or stars). Members can create their own goals, edit goals, mark goals as complete, connect to forums relating to those goals, and see progress in chart form on their goals. The system tracks progress on goals for use in incentive tracking (many companies reward health, productivity and well-being behaviors with money, gifts, and savings on health insurance coverage, and the system and method serves as a management tool for such programs).

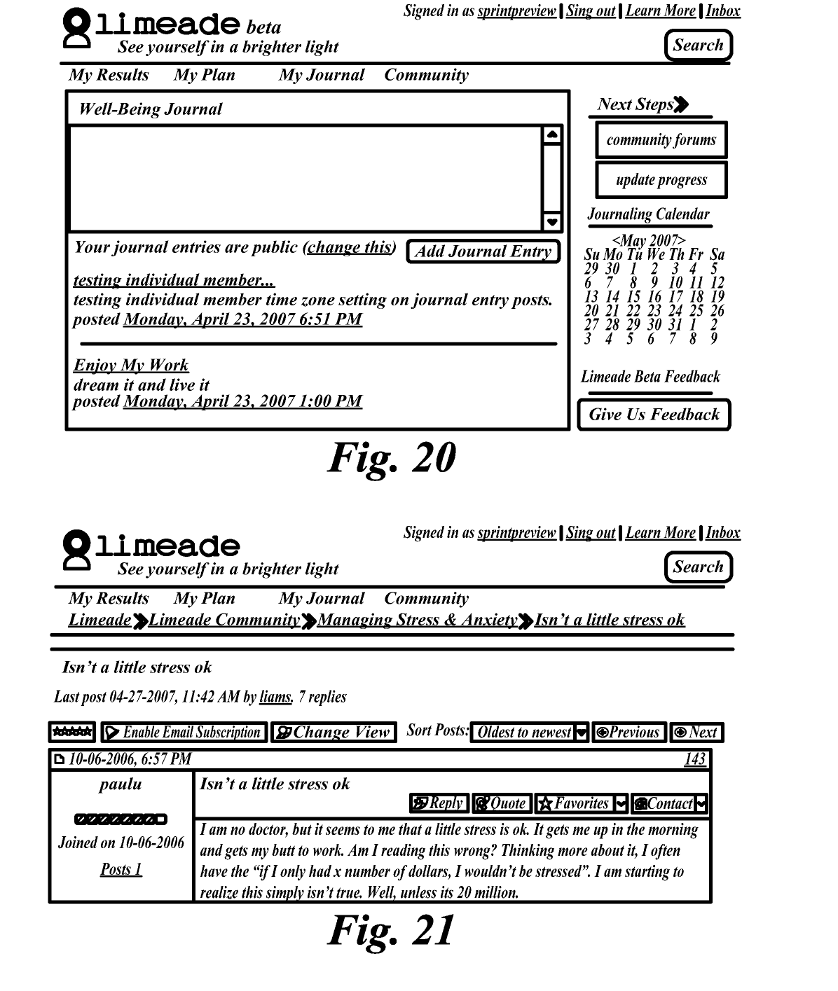

FIG. 20 shows a screenshot a well-being journal in one embodiment. The system provides a journal for members to enter their results in and choose to keep private or share with the community.

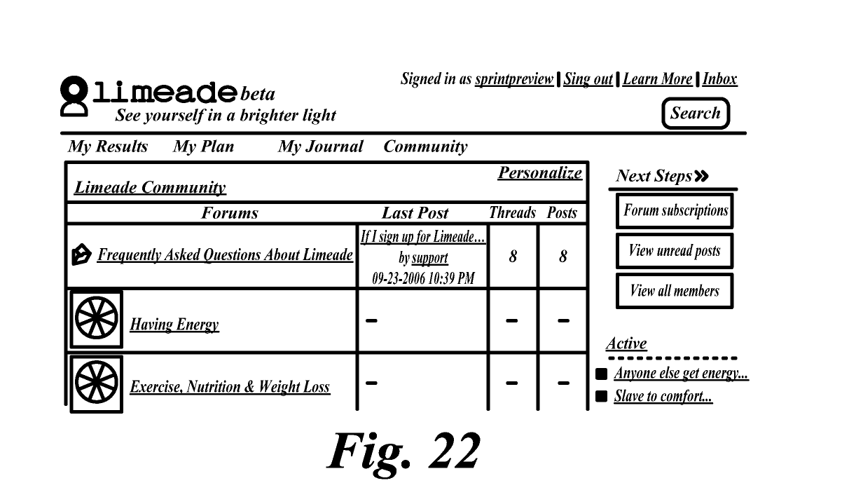

FIGS. 21-22 show screenshots of community forums used in one embodiment.

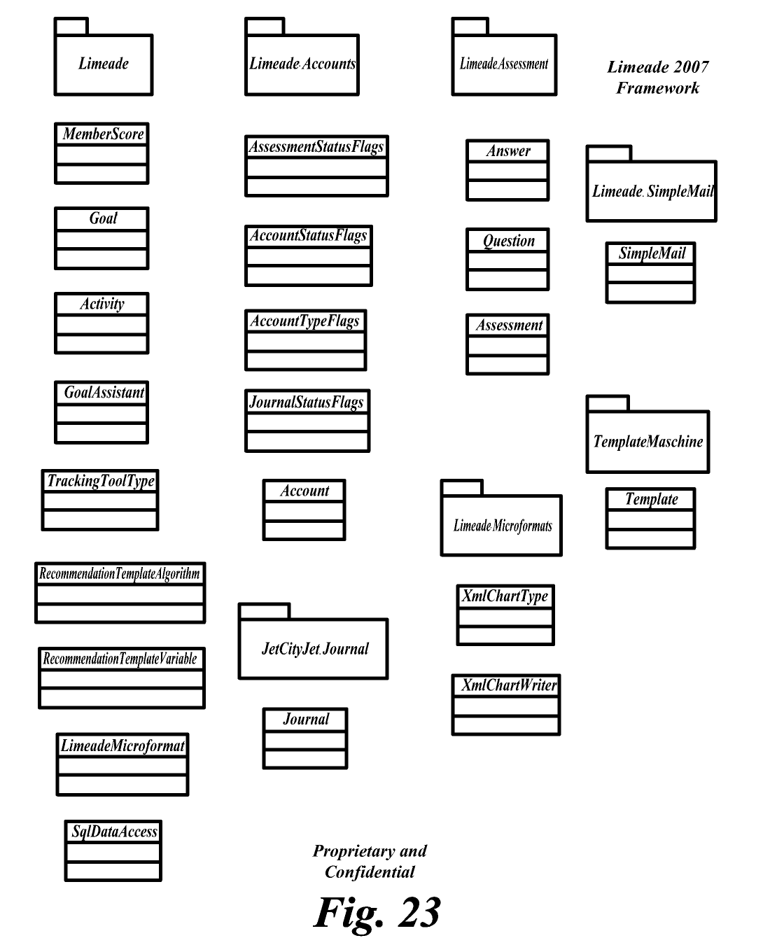

FIG. 23 shows an overview of the system framework. The system integrates employers, providers and end members based on the displayed framework.

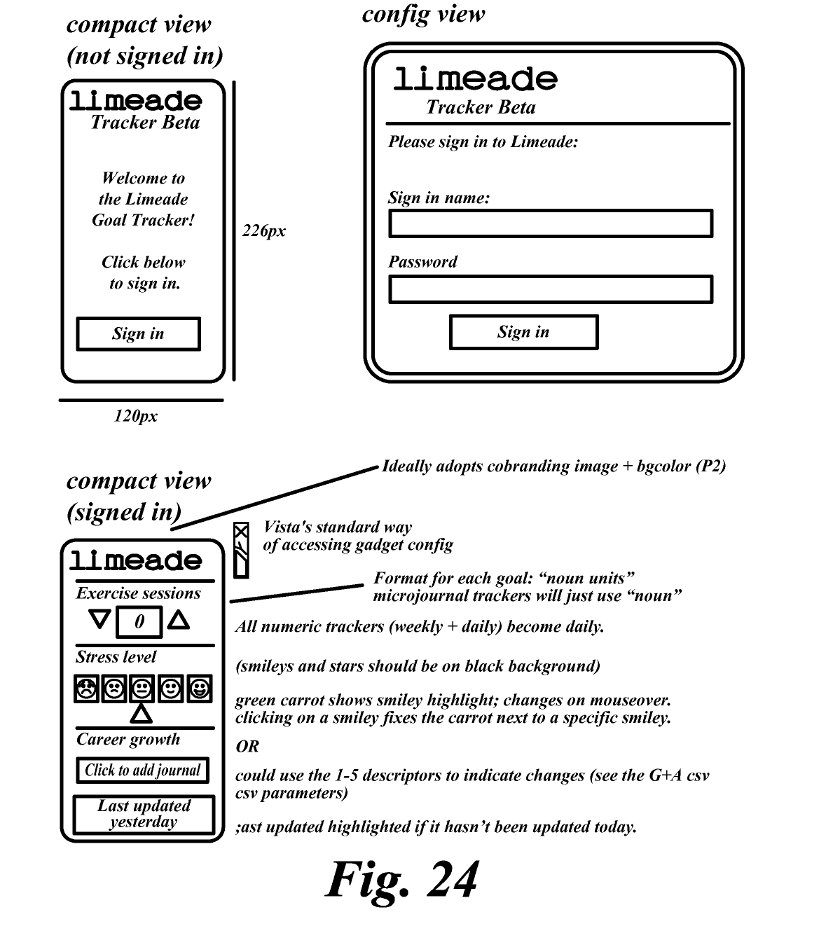

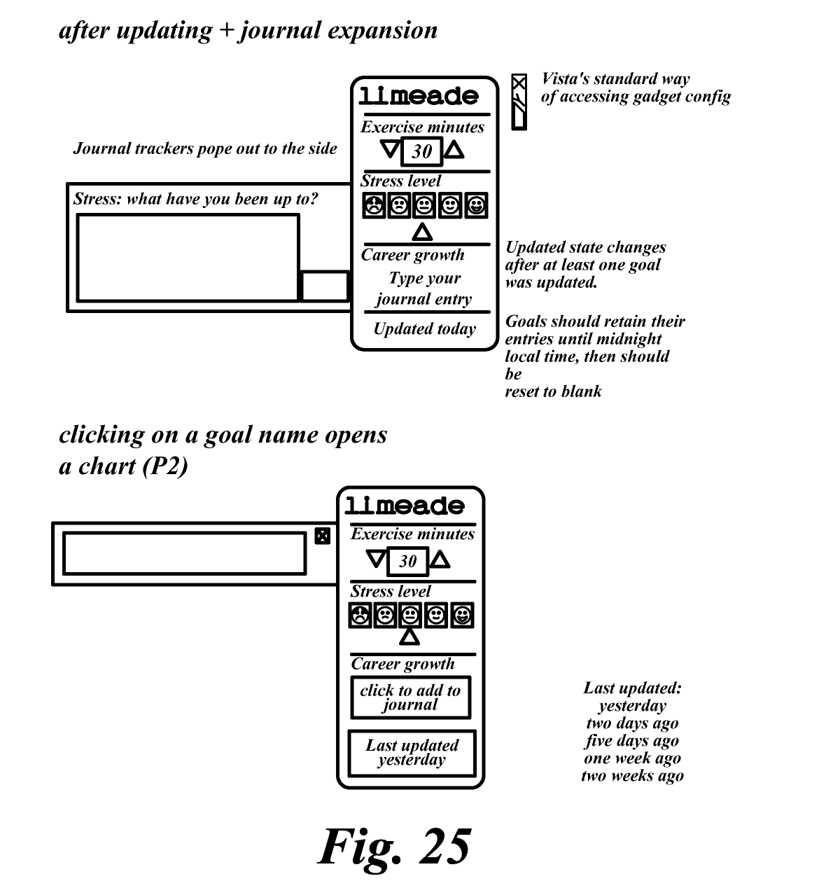

FIGS. 24-25 show screen shots of tracker widgets. In one embodiment, tracker widgets are desktop gadgets/widgets for ongoing tracking without having to visit a site. It allows a member to track progress through a desktop module.

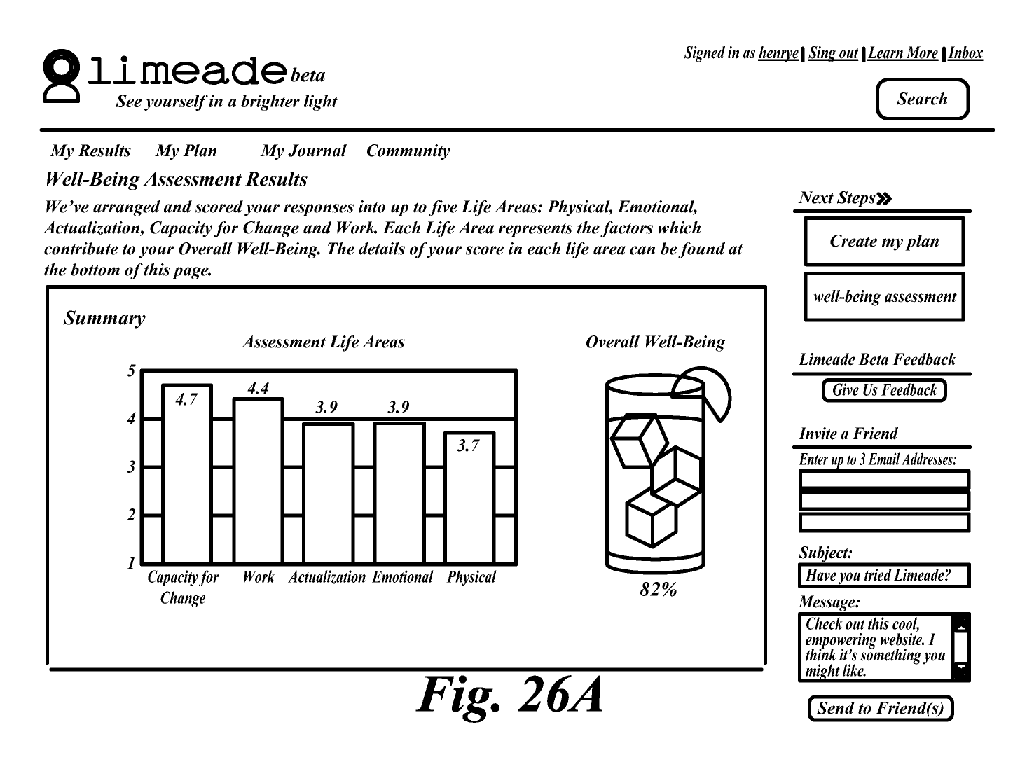

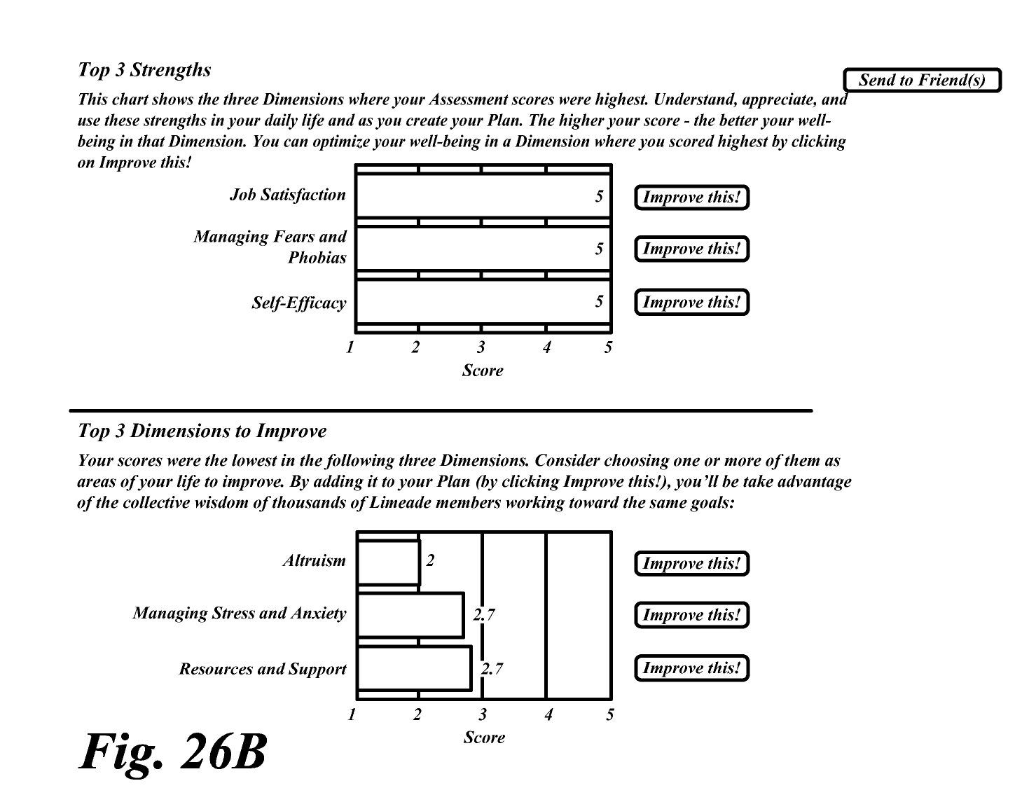

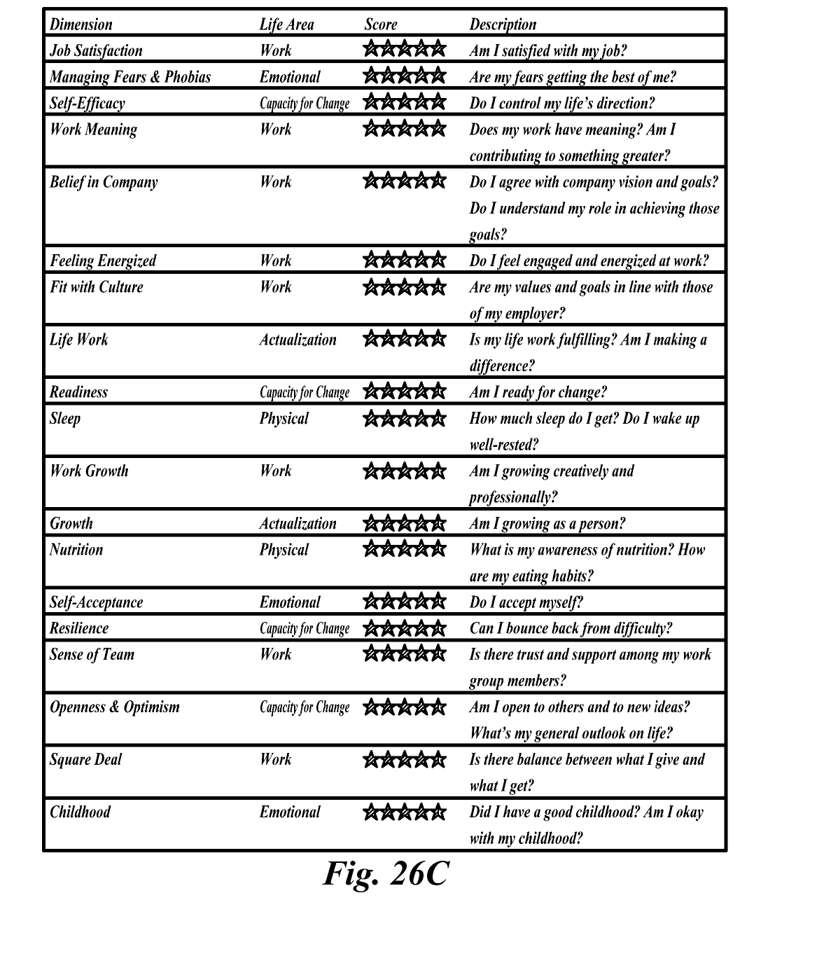

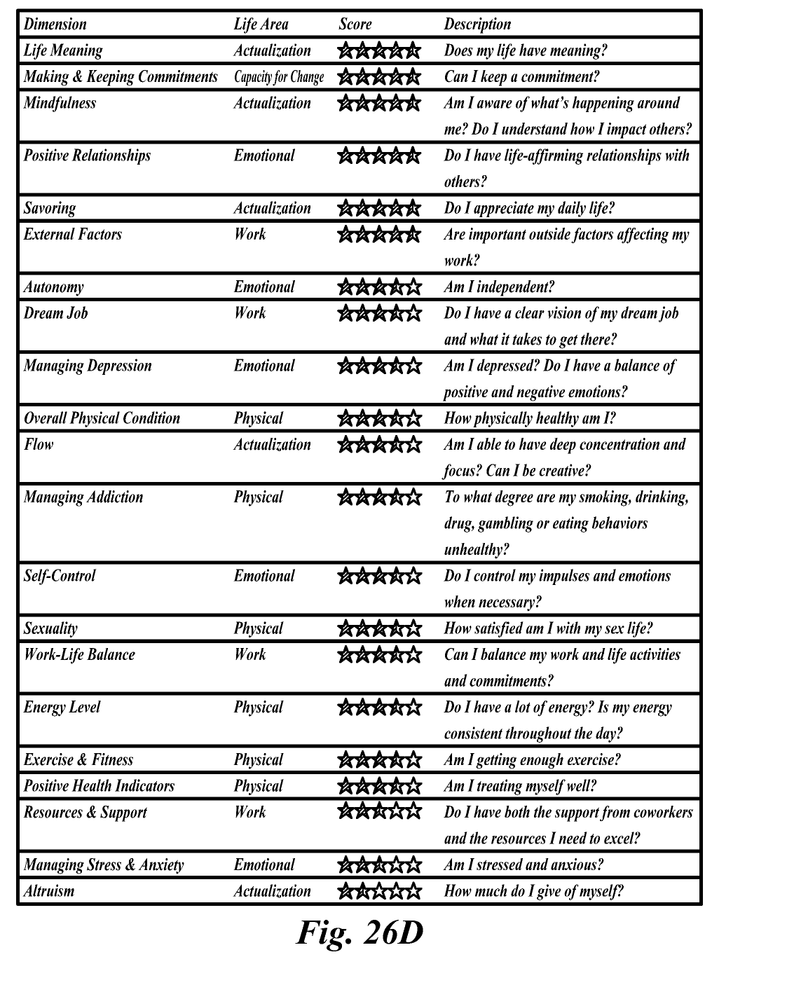

FIGS. 26A-26D show screenshots of a member's assessment in one embodiment.

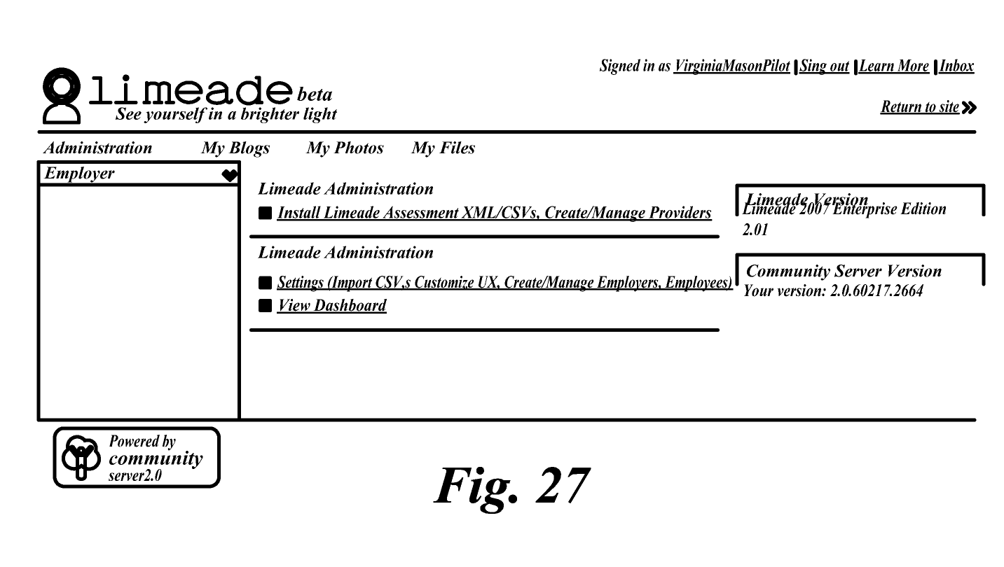

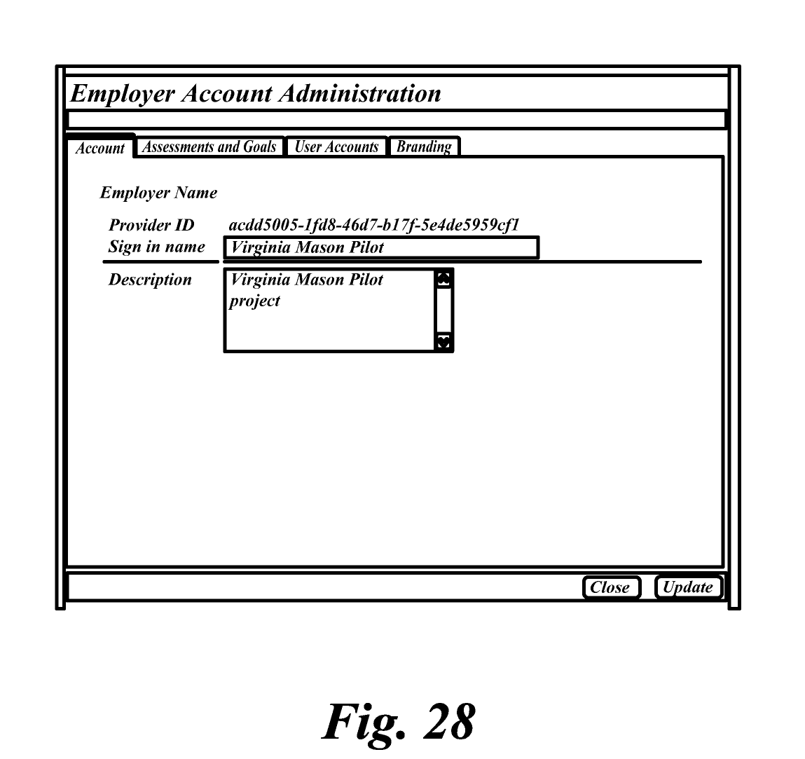

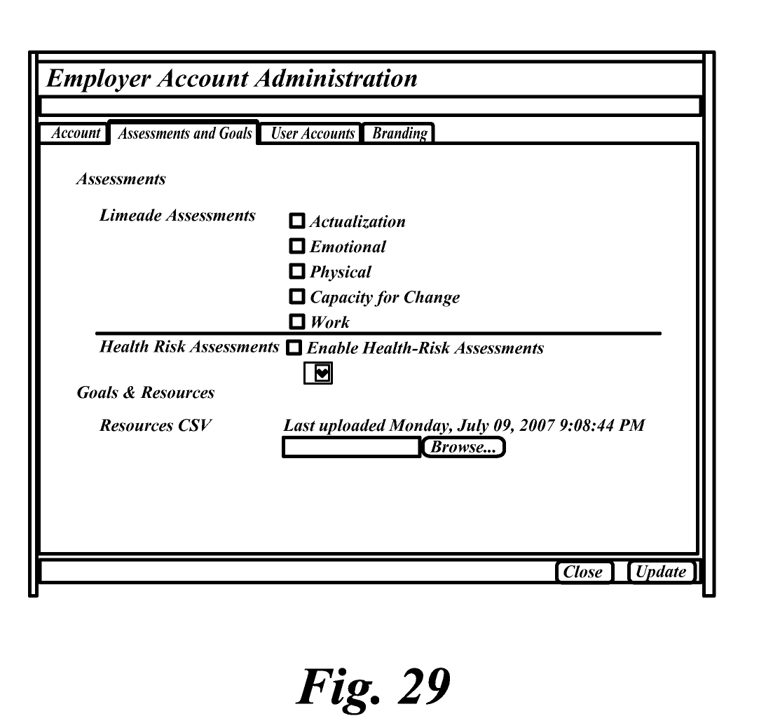

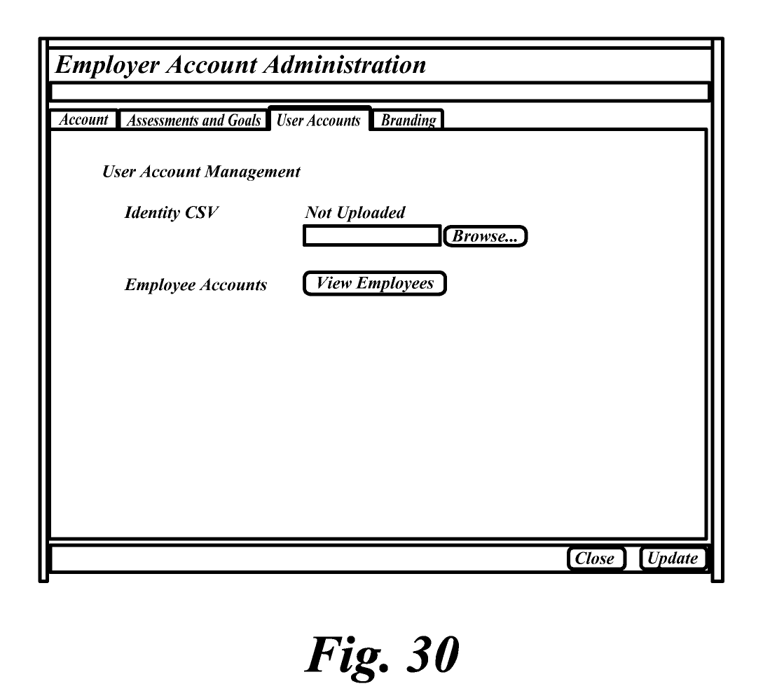

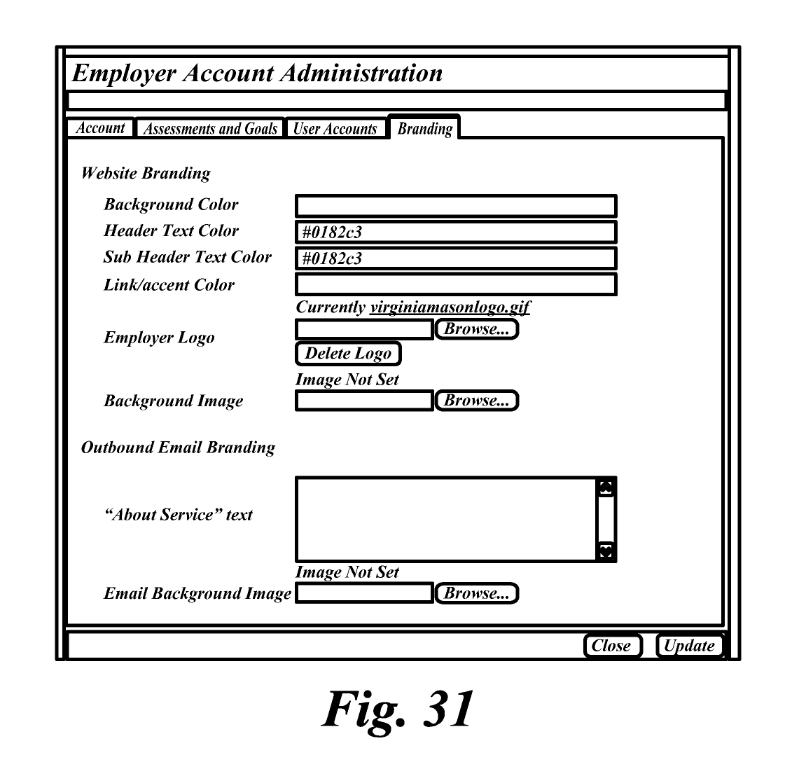

FIGS. 27-31 show screenshots of the steps an organization takes when launching the system and method.

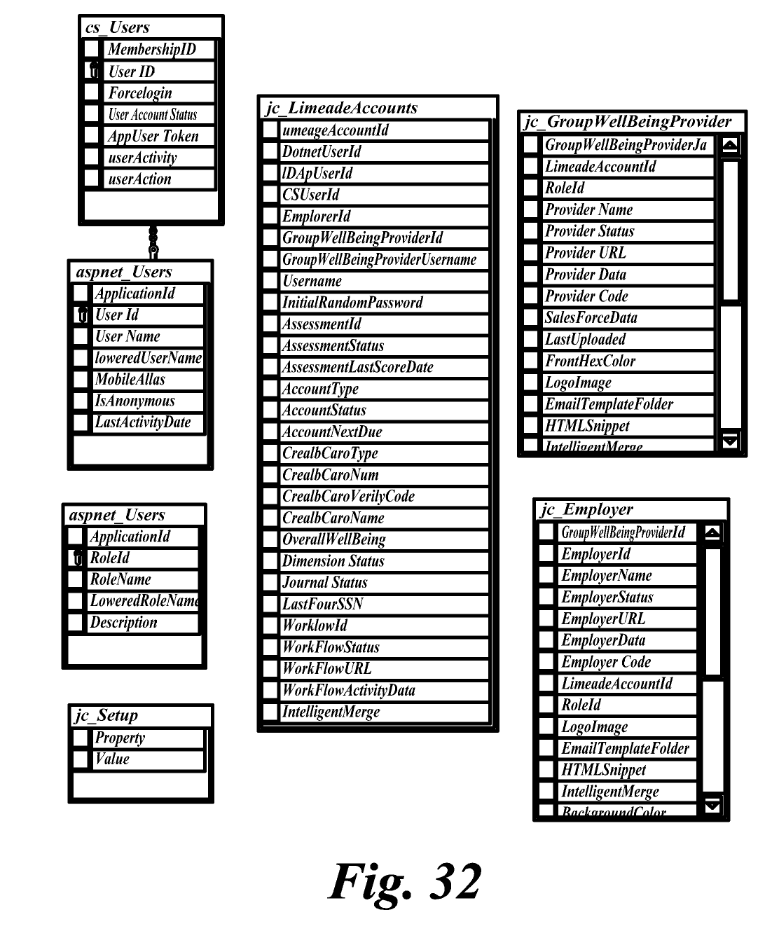

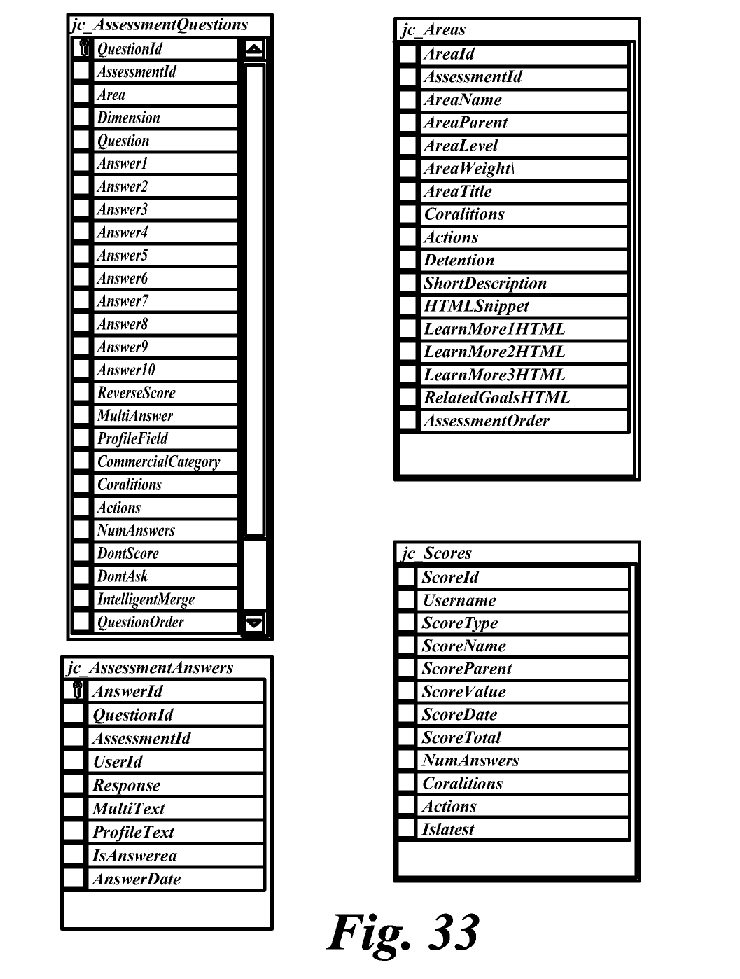

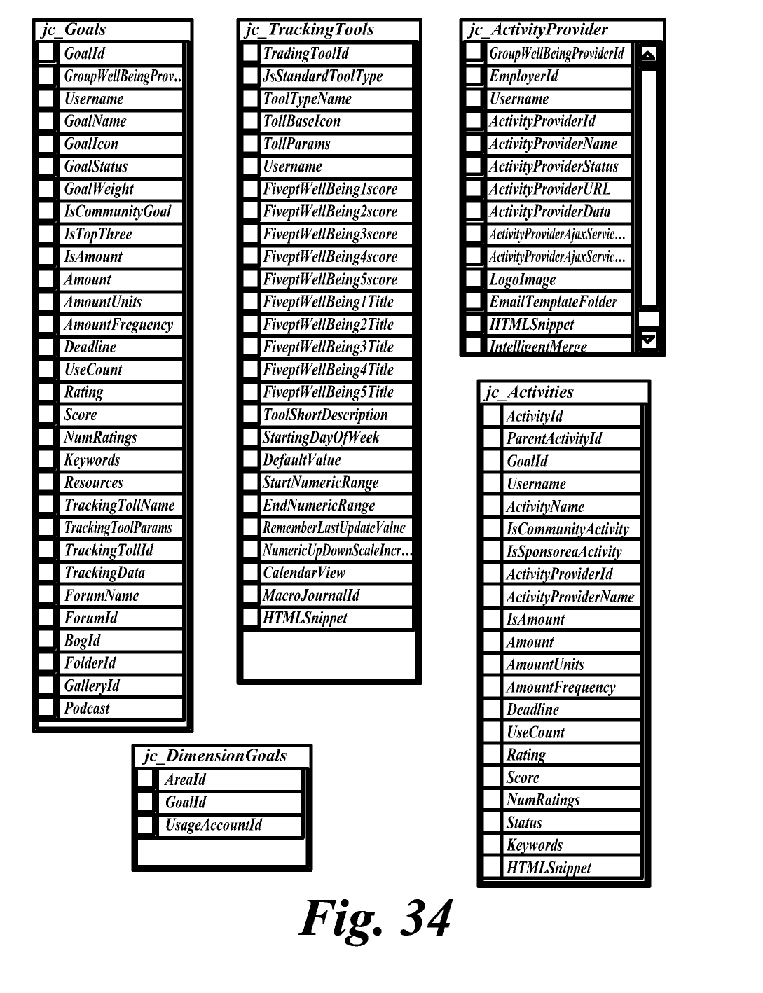

FIGS. 32-34 show screenshots of database relationships used in one embodiment of the system.

### Alternate Embodiments of the Present Invention

In accordance with an alternate embodiment included herein, there is provided a holistic, statistically correlated well-being assessment system and method.

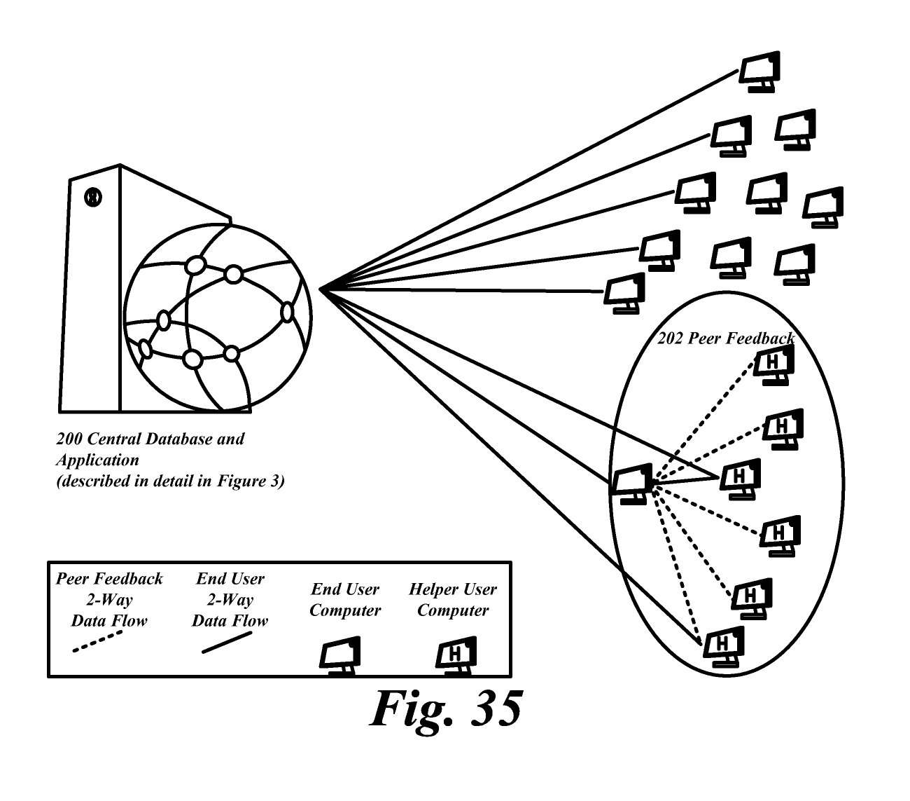

FIG. 35 illustrates a high-level System Topology of the software system that can deliver a holistic well-being assessment. The Central Database & Application 200 indicates a central processing center that can serve multiple end members. It lists the functional areas intrinsic to the application.

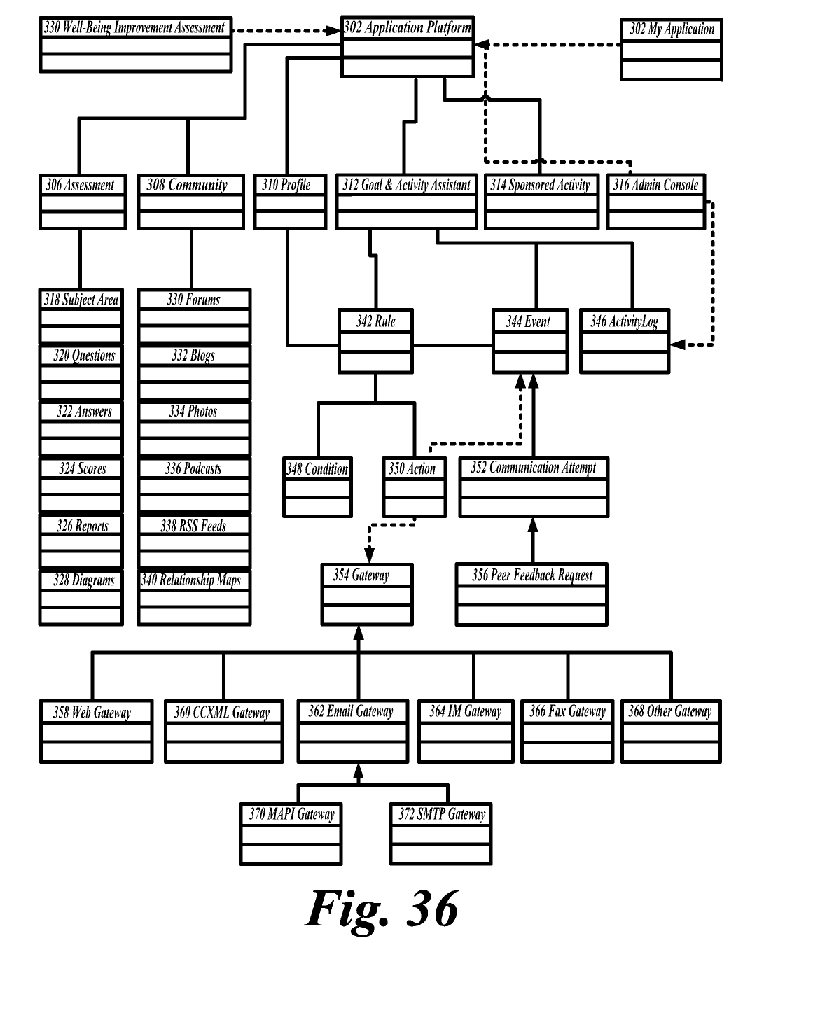

FIG. 36 illustrates functionality of invitation and interaction. This houses all of the functionality described in FIG. 35. Some end members can invite and interact with other end members, as shown in the Peer Feedback 202. Peer Feedback 202 is the functional area of the application that allows members to invite peers or friends to provide them with feedback on selected Sub-Areas of their Well-Being Improvement. In other words, whether web-based or not, the system can leverage the information of multiple members such that every individual member benefits from analyses performed across the data of all members. Details of technical architecture, describing specific algorithms, and analyses and functions are described in detail in FIG. 35. FIG. 35 illustrates Technical Architecture for Central Database & Application 200. The system as a whole delivers all functionality described herein to end-members. The function of each individual part is described in detail with the Figure. In general, the system takes members' Answers 322 to be assessed in Questions 320, and performs calculations on them that allow visual Reports 326 of the information.

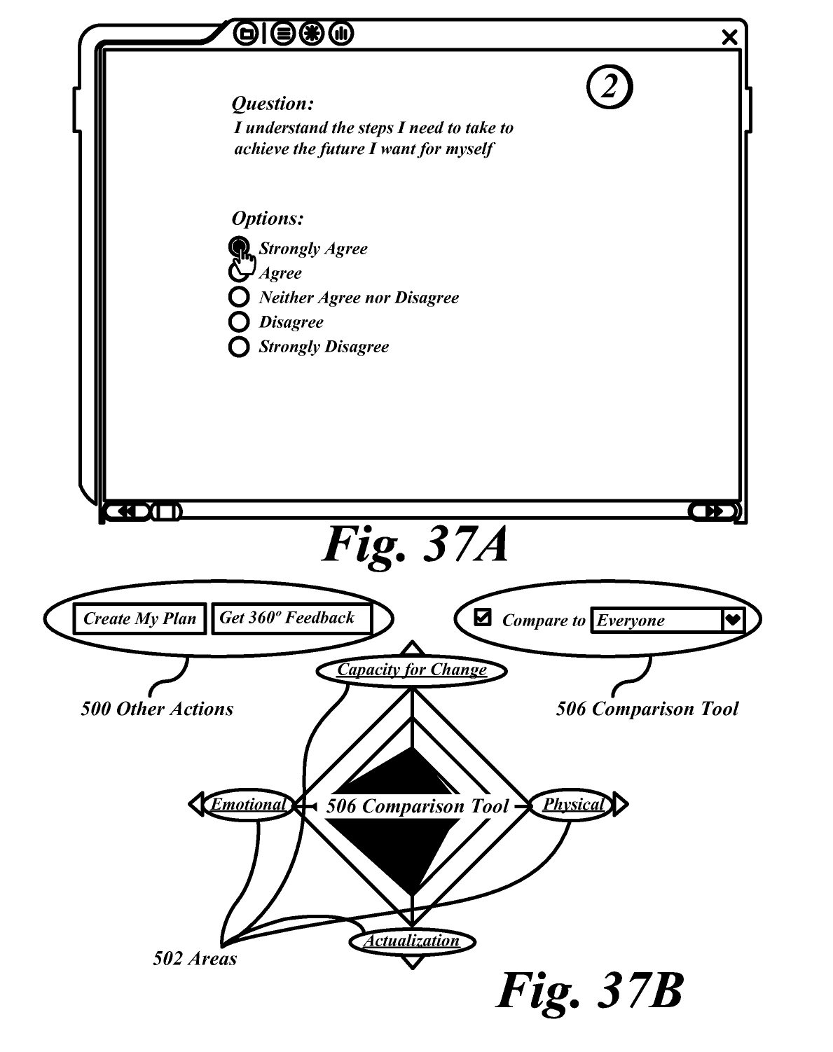

FIG. 37A illustrates a 5 point response scales in a screen shot view of a member survey. The member enters responses to a series of questions (in this figure but not necessarily always) in multiple choice (usually five point scale) format, and the responses are recorded by the system for use in other areas of the solution.

One key element to the entire system is the translation of member responses to Reports 326. In order to present consistent visual reports, the system generally relies on standardized five-point response scales. Each member Answer 322 can be recorded as a numerical value from one to five. Where this is not possible (e.g., in some cases N/A, multiple possible answers (checkbox or radio button), textual and binary questions are preferred), Answer 322 values may be translated to a five point scale equivalent. Numerical values for all Answers 322 within each Sub-Area (as described in FIG. 1) can be weighted and averaged to calculate and present one Sub-Area score, as visually represented in FIG. 37B. The weighting can vary over time as certain factors are determined by analysis to more or less define the Sub-Area. These Sub-Area averages can in turn be averaged to determine an overall Area average, as visually represented in FIG. 37A. The weighting can vary over time as certain factors are determined by analysis to more or less define the Area. These Area averages may be weighted and averaged to present one overall Well-being average. The weighting can vary over time as certain factors are determined by analysis to more or less define Well-Being.

Over time, if certain Sub-Area Questions 320, the Answers 322 are shown to define any other Sub-Areas, either individually or in combination with other Answers 322, such Answers 322 may be included in calculations of other Sub-Areas (in addition to or instead of the original Sub-Area.) Likewise, if any Sub-Areas define any other Areas, those Subarea Scores 324 may be included in calculations of other Areas.

The statistical validity of the Assessment 306 is ensured by adherence to content and face validity processes. The Assessment 306 is designed to reduce social desirability/gaming in member responses, specifically by: a) being self-focused (not employer, insurer- or professional focused), b) having no specific, desirable end-point (self-improvement is a perpetual process), c) tracking everyone's improvement regardless of their Assessment 306 results, d) incorporating Peer Feedback (e.g., from Feedback Request 356 or from hitting the “Get 360° Feedback” widget of FIG. 37A) via 3600 data gathering, e) covering as much of the Area and Sub-Area as possible, so that one Question 320/Answer 322 can not solely determine someone's Score 324 in that area, f) alternating the phrasing of Questions 320 (e.g., sometimes wording items negatively), g) not overtly stating what we are measuring (i.e., not showing the Sub-Area name) during the Assessment 306, and h) removing questions that test experts deem socially desirable (based on professional reviews).

Well-Being Improvement Assessment 300 lists a set of individual end member responses to the Well-Being Improvement Assessment as described conceptually in FIG. 1, and as represented in example form in FIG. 36. This is the first application of the Application Platform 302, and represents at a high level that member data is collected, stored, analyzed and otherwise used in the technical infrastructure of the Application Platform 302. [Para 54] Application Platform 302 is term used to describe all of the other elements described in FIG. 3 and their interactions. The application platform is extensible to other modules, operations and any number of other functions. It also represents a platform for future products and extensions, e.g., new Assessment 306 types. The term ‘platform’ is chosen because it implies a generic and extensible approach to applications, communications, assessments and other data interactions. My Application 304 is the Area of the application that is specific to an individual member, including all of their specific assessment results, plans, recommendations, community settings, etc. It represents a member-specific view.

The Assessment 306 is the generic assessment functionality. This is the delivery mechanism of the Well-Being Improvement Assessment 300, and may be applied to any other future assessments. It contains all of the components listed vertically below it in the Figure, specifically Subject Area 318, Questions 320, Answers 322, Scores 324, Reports 326 and Diagrams 328. It communicates bi-directionally with the Application Platform 302, e.g., sending Assessment data to the platform for storage, analysis or the creation of Events 344 through the Goal & Activity Assistant 312 and by receiving information from the Application Platform 302 about what Assessments to offer the member. Subject Area 318 is the part of Assessment 306 that describes the subject area of any assessment or assessment section.

Questions 320 are a component of Assessment 306 that stores and manages questions of any assessment. Answers 322 are a component of Assessment 306 that stores and manages answers of any assessment. Scores 324 is a component of Assessment 306 that stores, applies and manages numerical values based on member responses, question weightings and other factors, individually and in combination. Scores 324 is used in Reports 326, recommendations from the Goal & Activity Assistant 312 and other areas of the Application Platform 302. Reports 326 is a component of Assessment 306 that describes visual representation of scores in various media to end members, and programmatic distribution of visual and non-visual scores to systems or members. It stores reports, report parameters, visual representations or access thereto, and data and metadata. Diagrams 328 are a component of Assessment 306 that represents Scores 324 using various graphical notations, including charts, and directed and non-directed graphs. Community 308 is a function for connecting system members and allowing them to access and share information in various media. It is the place in the system where people and their data connect, and contains all of the components listed vertically below it in FIG. 35, specifically Forums 330, Blogs 332, Photos 334, Pod casts 336, RSS Feeds 338 and Relationship Maps 340. It communicates bi-directionally with the Application Platform 302, e.g., sending Blogs 332 to the Application Platform for storage or analysis, and leveraging Profile 310 and possibly many other architecture components to determine how the Application Platform 302 can respond to Community-related member actions.

Forums 330 are a component of Community 308 that allows members to discuss topics of mutual interest with each other. Blogs 332 is a component of Community 308 that allows members to maintain their self-improvement journals and/or discuss topics with other members. Photos 334 are a component of Community 308 that allows members to share visual images with each other. Pod casts 336 is a component of Community 308 that allows members to share media with each other. (‘Members’ is a generic term and includes the inventor. ‘Podcasts’ is used here as a generic term and may describe recordable media of various protocols, sponsors or technologies). RSS Feeds 338 is a component of Community 308 that allows members to send and receive information with each other. RSS is a family of web feed formats, specified in XML and used for Web syndication. RSS is used by (among other things) news websites, “weblogs” and “podcasting”. (Definition from Wikipedia: The abbreviation ‘RSS’ is variously used to refer to the following standards: Really Simple Syndication (RSS 2.0), Rich Site Summary (RSS 0.91, RSS 1.0), RDF Site Summary (RSS 0.9 and 1.0.) Web feeds provide web content or summaries of web content together with links to the full versions of the content and other metadata. RSS, in particular, delivers this information as an XML file called an RSS feed, web feed, RSS stream, or RSS channel. In addition to facilitating syndication, web feeds allow a website's frequent readers to track updates on the site using an aggregator).

Relationship Maps 340 is a component of Community 308 that allows members to see visually how they are connected with other members. IProfile 310 is a comprehensive list of a member's identification information. IProfile can be used in various ways, and in combination with various other pieces of data to trigger system actions, communications and other interactions. In a simple sense, IProfile communicates with Rule 342 and the Application Platform 302. In a more nuanced sense, Profile describes the things about members or data that are pertinent and can be acted on in some way. For example, imagine a male member who likes getting email but hates getting faxes, who has finished twenty of the twenty four components of a Well-Being Improvement Assessment 300 (and therefore sees incomplete Assessment Results, Sub-Area & Sub-Area Details Views as described in FIG. 37B). This situation may trigger an e-mail reminder through the Email Gateway 354 (a specific Event 350), based on a rule and his profile. E.g., the rule may say anyone in this state who requests such reminders and has certain profile settings shall receive such an e-mail. Goal & Activity Assistant 312 is the system that recommends specific courses of action to a member based on various factors uncovered via the assessment and other accumulated system efficacy knowledge. It may make recommendations on member Goals (e.g., “Sign up for a course on Stress Management”) based on Assessment 306 (e.g., a low Anxiety & Stress 126 score), Community 308 (e.g., a Podcast 336 they joined on Stress Reduction [not shown]), Rule 342, Event 344, or some combination of these.

Sponsored Activity 314 is a place to store and a means to manage all recommended courses of action from other companies and company affiliates and advertisers. It leverages data in the same way Goal & Activity Assistant 312 does. Admin Console 316 is a company-internal tool to allow company members to manage various parts of the application platform. For example, it manages Assessment 306, bulk imports of CSV and XML data into the Central Database & Application 200, and it tracks member preferences, e.g., for communication methods, privacy settings, and email addresses. Rule 342 describes a logic center of the platform, a place where combinations of various factors drive what is presented to which members, when, where, and how. Event 344 is a system or member occurrence that can be recorded by the system. For example, an Event 344 can be a) a member generating a Report (Reports 326), b) the system receiving a Communication Attempt 352 such as a Peer Feedback Request 356, c) the recording of an Answer 322 to a Question 320, d) the generation of a recommendation by the Goal & Activity Assistant 312, or others. Activity Log 346 allows the system to track and report on a history of Events 344, Actions 350, Rules 342, and other information. Condition 348 describes a Boolean expression as part of a Rule 342, which, if true, executes one or more Actions 350. For example, the Action 350 to generate Reports 326 only executes for a Sub Area (from the Sub-Area List 102) if the Condition 348 is “90% or more of the responses for that Sub-Area have recorded answers 322.” With each Condition 348, the following operators may be used: a) ‘=’ means “equals”, b) ‘< >’ means “does not equal”, c) ‘>’ means “greater than”, d) ‘<’ means “less than”, e) ‘>=’ means “greater than or equal to”, f) ‘<=’ means “less than or equal to.” Action 350 describes something happening to data within the Application Platform 302 as part of a Rule 342. Actions may occur in conjunction with a Condition 348 to trigger an Event 344. They may be system or member-generated. Communication Attempt 352 describes an effort to communicate with a member or another system triggered by an Event 344.

Peer Feedback Request 356 is an example Communication Attempt 352 that tracks member-initiated communication attempts to peers, whether known or anonymous, as describes with regard to Comparison Tool 506, 606 in FIGS. 6 and 7 below. Gateway 354 is a generic vehicle for prioritizing and routing specific example communication methods and media. It is a superset of all possible gateways, including example gateways listed below it in the Figure, Web Gateway 358, CCXML Gateway 360, Email Gateway 362, IM Gateway 364, Fax Gateway 366, Other Gateway 368, and the two example Email gateways MAIPI Gateway 370 and SMTP Gateway 372. Web Gateway 358 describes an example Gateway 354 to other web services. CCXML Gateway 360 describes an example Gateway 354 to CCXML. (Definition from Wikipedia: Call Control eXtensible Markup Language (CCXML) is an XML standard designed to provide telephony support to VoiceXML. Where as VXML is designed to provide a VUI interface to a voice browser, CCXML is designed to inform the voice browser how to handle the telephony control of the voice channel. The two XML applications are wholly separate and are not required by each other to be implemented). Email Gateway 362 describes an example Gateway 354 to email (e.g., to MAIPI and SMTP.) MAIPI Gateway 370 describes an example Email Gateway 362 and SMTP Gateway 372 describes an example Email Gateway 362. IM Gateway 364 describes an example Gateway 354 to Instant Messaging. Fax Gateway 366 describes an example Gateway 354 to Facsimiles. Other Gateway 368 describes an example Gateway 354 that may include any other or future communication gateways.

FIG. 37B illustrates Assessment Results and Area View. This shows that all of the Well-being Model information entered by the member can be presented in visual form. This view is generated via the Reports 326 and Scores 324 function described in FIG. 35. (Reports 326 and Scores 324 calculations in the Database populate a reporting engine). The member at his/her option can be able to compare his/her own results via the Comparison Tool 506 vs. the results of various populations and benchmarks, including but not limited to all other assessment takers, assessment makers his/her age and sex, established norms, and others' perceptions of him/her.

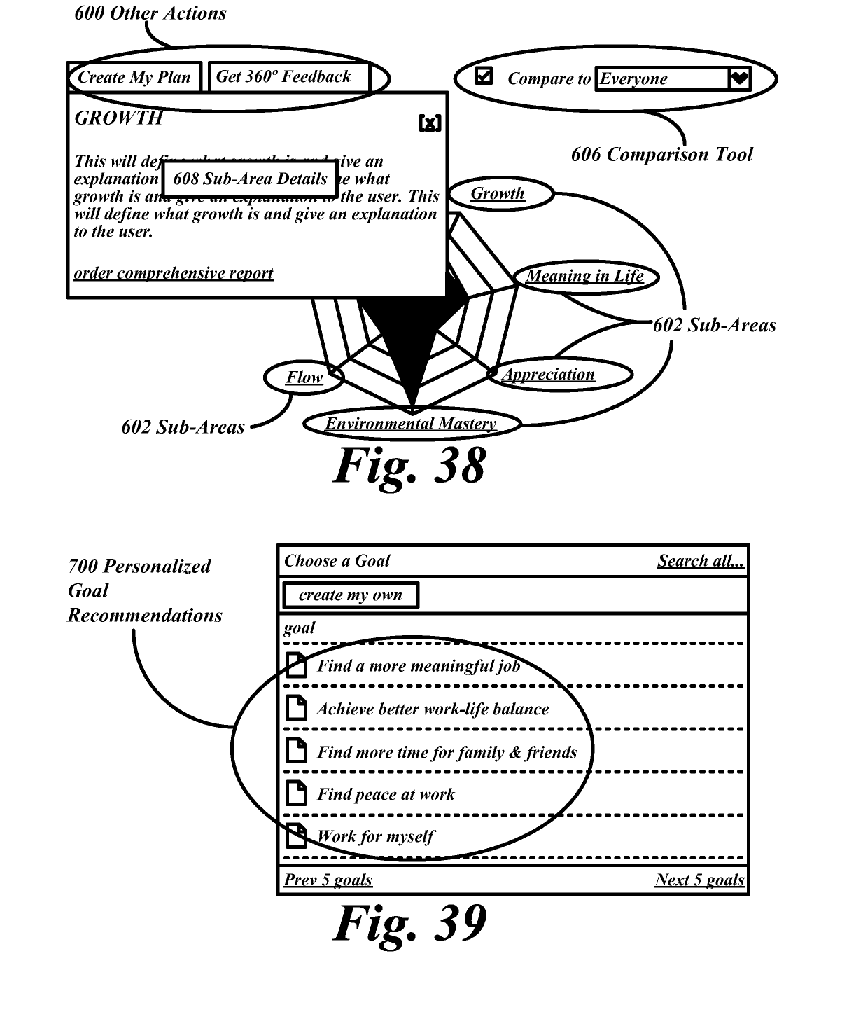

FIG. 38 schematically illustrates assessment Results, Area View to get to Assessment Results, Sub-Area and Sub-Area Details Views. Other Actions 500 gives example representation of actions (other than a member reviewing his/her own results) that the member can take from this functional area. FIG. 38 also illustrates Assessment Results, Sub-Area and Sub-Area Details Views. This shows that all of the well-being model information entered by the member can be presented in visual form. The member at his/her option can be able to compare his/her own results via the Comparison Tool 606 vs. the results of various populations and benchmarks, including but not limited to all other assessment takers, assessment makers his/her age and sex, established norms, and others' perceptions of him/her. Text descriptions and analysis of his/her results can be present. Members can navigate back to Assessment results, Area View via standard web navigation methods. Other Actions 600 is an example representation of actions (other than reviewing their own results) that the member can take from this functional area.

FIG. 39 illustrates the Create My IPlan example link leads to a set of member-specific Goal Recommendations, and the ‘Get 360° Feedback’ button leads to an invitation mechanism. FIG. 39 also illustrates a goal recommendation view and a personalized Goal Recommendations 700. This helps the member find Goals that are highly likely to lead to real Well-Being Improvement. The algorithm variables and their relation to one another are described in the Figure. The recommendations are stored in the Database as part of the member's profile. The Goal Recommendations 700 describes member-specific, personalized goal recommendations based on the various member scores. The algorithm that calculates these recommendation can be expressed as a simple function as follows:

Goal Recommendation for goals (1-n)=(member score on Sub-Area 1) (weighting for Sub-Area 1)+(member score on Sub-Area 2) (weighting for Sub-Area 2)+ . . . +(member score on Sub-Area n) (weighting on Sub-Area n.) The weightings of the various Sub-Areas accounts for the likelihood of anyone to improve that Sub-Area or combination of various Sub-Areas, the likelihood of the person with a particular combination of scores to improve that Sub-Area, the availability of useful Activities to improve that Sub-Area, recommendations and ratings of other System members, including those whose IProfile resembles the member's, and member preference. The goal recommendation system is extensible to other factors in the future.

The feedback button allows a member to select assessment Areas and/or Sub-Areas, and then select people to whom the invitation can be sent. After the end members answer the questions about the inviting member, the member can see his own self-assessment in comparison to the assessment of him/her provided by others. He/she can also see comments from fellow invitees. Areas 502 is an example representation of Areas from the Area List. Graph 504 is an example representation of graphical view of Assessment 306 Reports 326/Scores 324. Comparison Tool 506 is an example representation of a widget that provides a comparison of a member's assessment results with those about the member provided by his peers, and with other groups. The Comparison Tool 506 has a simple drop-down menu that can allow the member to choose the population or benchmark to which he/she is comparing his own self-generated assessment results. The two sets of results can be displayed visually as two sets of data values in the same Area Graph 504, in the Assessment Results, Area View and in the Assessment Results, Sub-Area and Sub-Area Details View. These views are described in FIGS. 5 and 6 above that illustrates a comparison.

The Create My Plan example link would lead to a set of member-specific Goal Recommendations FIG. 39, and the ‘Get 360° feedback’ button would lead to an invitation mechanism (not shown), wherein someone could select assessment Areas and/or Sub-Areas, and then select people to whom the invitation would be sent. After the end members answer the questions about the inviting member, the member could see his own self-assessment in comparison to the assessment of him/her provided by others, AKA “Peer Feedback” as described in FIG. 4 above. Peer Feedback can include comments and other information or media from his invitees.

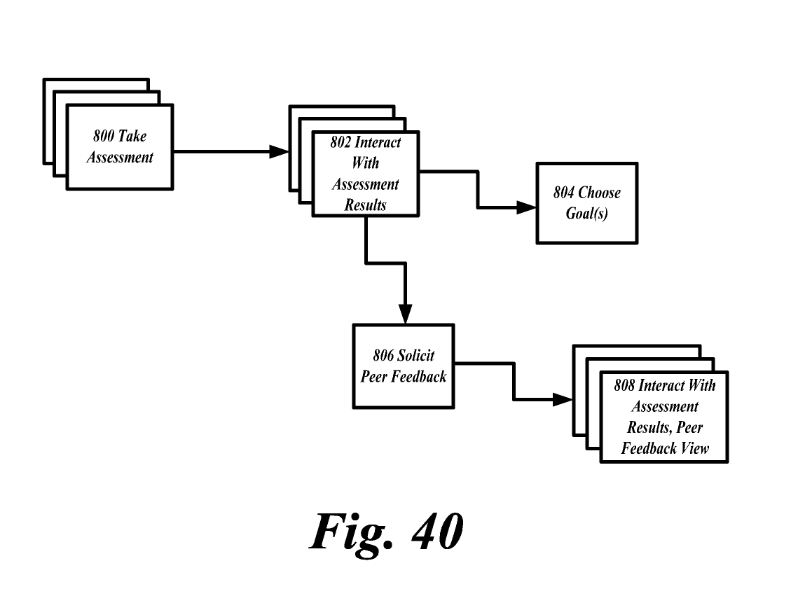

FIG. 40 illustrates peer feedback functionality and a Member Experience Flowchart. Both describe how a member can navigate the member experience described in the detailed descriptions of all other figures. Take Assessment 800 is the part of the member interaction scheme wherein the member completes an Assessment by answering questions, i.e., as illustrated visually by FIG. 5 below. This comparison tool works by either a) gathering all or some Assessment Results from peers as described in Peer Feedback 202 in FIG. 35 above, 356 in FIG. 35 above, and 806, 808 in FIG. 40 or by b) allowing the member to compare to various benchmarks or population averages from multiple end-member systems that meet the member-specified criteria, as shown at a high level in a detailed level in FIG. 36, specifically via Scores 324. Then the comparative Reports 326 are generated by the Assessment 306 and displayed visually.

The Interact with Assessment Results 802 is the part of the member interaction scheme wherein the member looks at and analyzes his/her results, e.g., as presented in FIGS. 37A and 37B. [Para 109] Choose Goal(s) 804 is the part of the member interaction scheme wherein the member reviews personalized Goal Recommendations as described in FIG. 39 above, and may select goal(s) to work on.

The Solicit Peer Feedback 806 is the part of the member interaction scheme wherein the member solicits Peer Feedback. Peer Feedback is the output of the ‘Other Actions’-‘Get 360 Feedback’ functionality described in FIG. 37B. Interact With Assessment Results 802, Peer Feedback View 808 is the part of the member interaction scheme wherein the member looks at and analyzes his/her results, e.g., as presented in FIGS. 37B and 38, specifically by using the Comparison Tool elements 506 and 606, and choosing the comparison set that reflects his/her peers opinions of him/her.

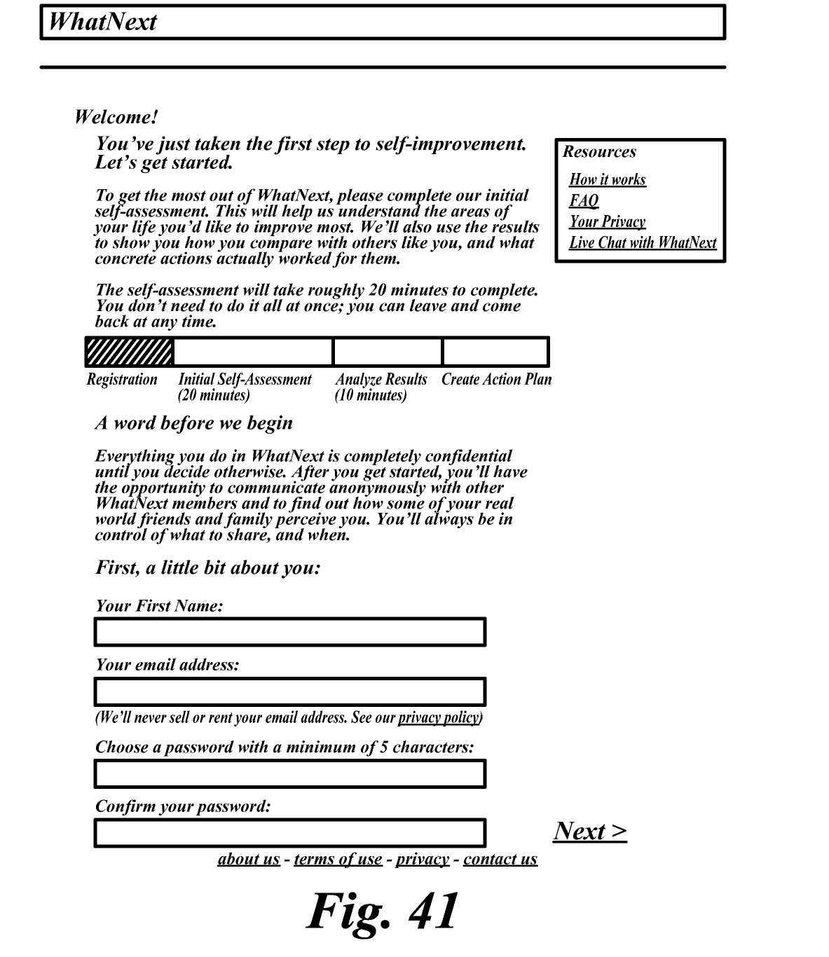

FIG. 41 shows a screenshot of a registration page. This page is a screen a member would see when preparing to take the assessments.

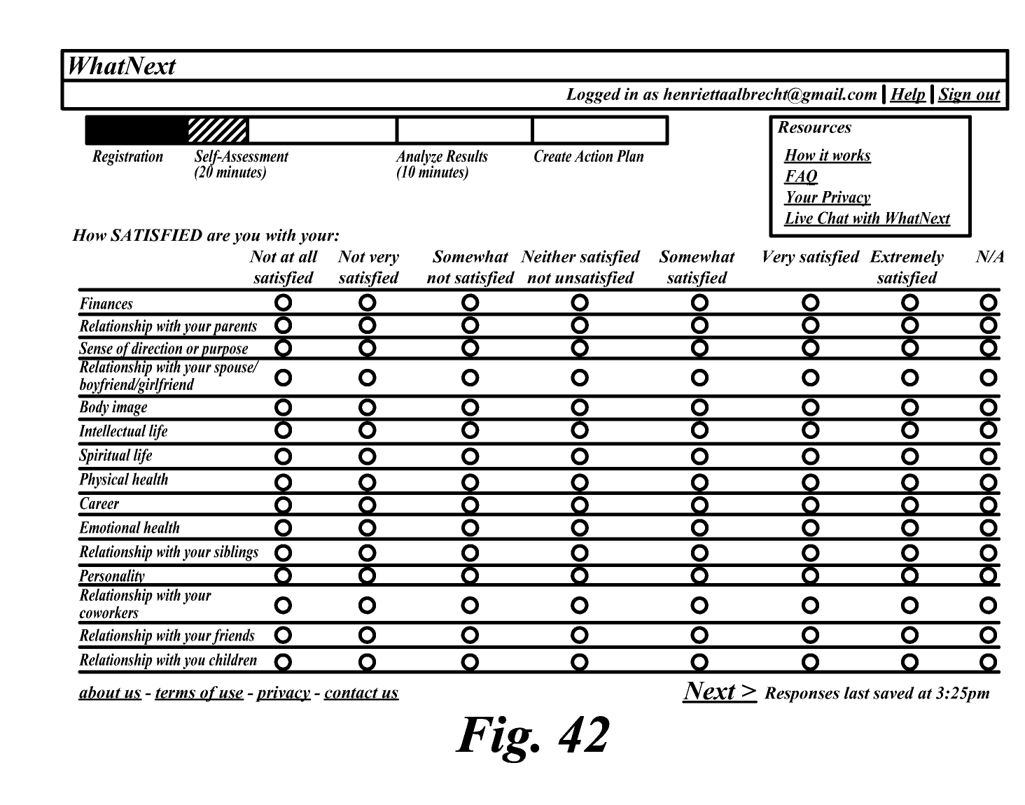

FIG. 42 shows a screenshot of an example assessment page. This page shows questions, a variety of topics and a numerical rating scale. A member, using the radio buttons, enters a response for each question. The response is stored in memory and processed by the recommendation algorithm.

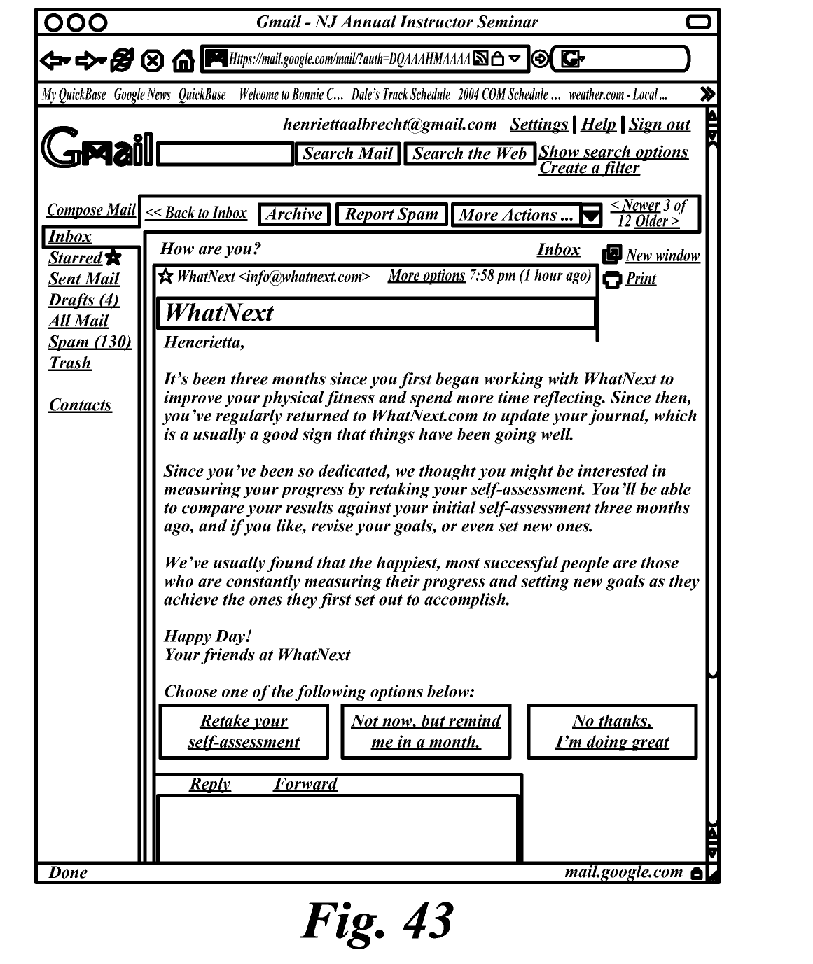

FIG. 43 shows a screenshot of email reminder. This email reminder is sent to a member inviting them to take the assessment over again after a period of time in order to update their results and further track progress.

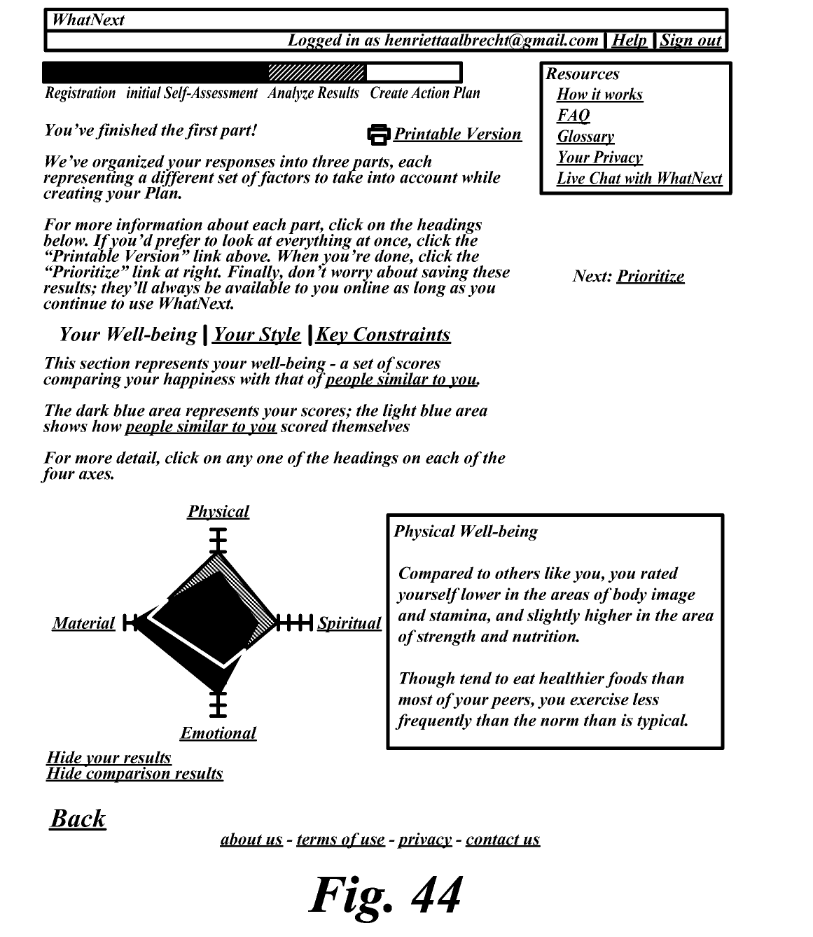

FIG. 44 shows a screenshot of a member's well-being. This screen shows a member their well-being scores and then compares those scores to member's similar to them. It also shows the member what areas the member was lower than their peers in and some reasons why.

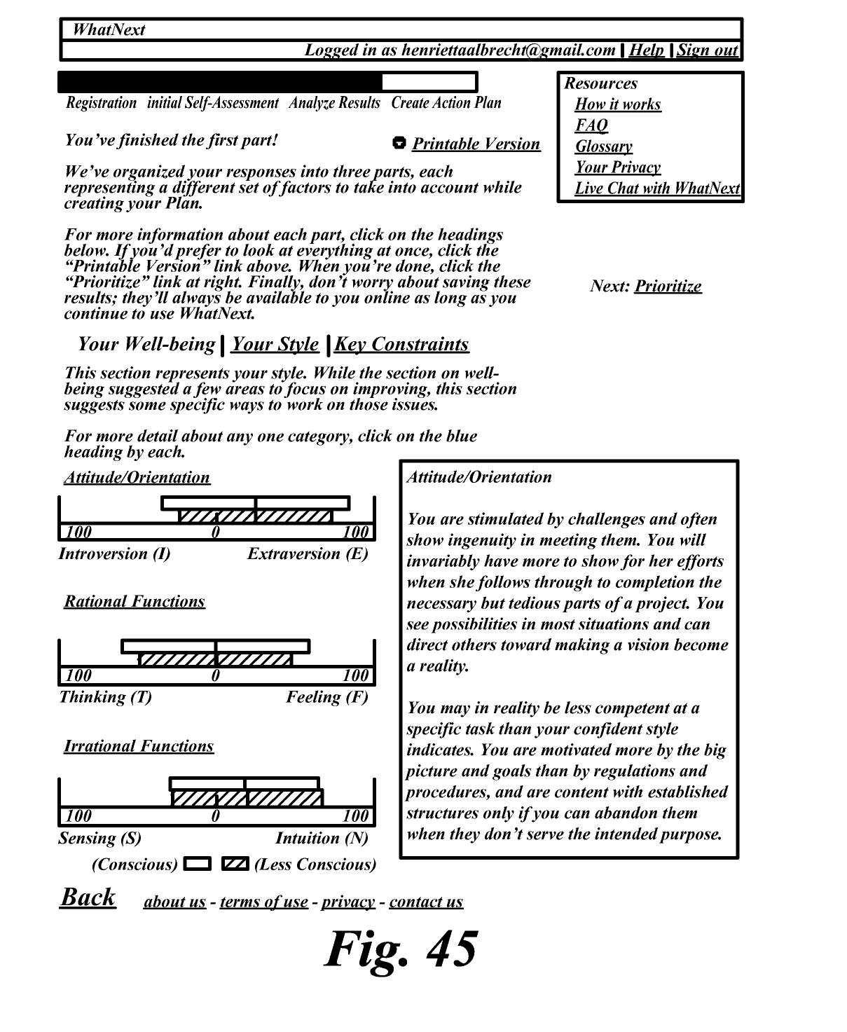

FIG. 45 shows a screenshot of a member's results. This screen shows a member individual factors that contribute to their well being. It shows the member how they scored and provides a brief synopsis of the factor and some specific ways to work on those issues.

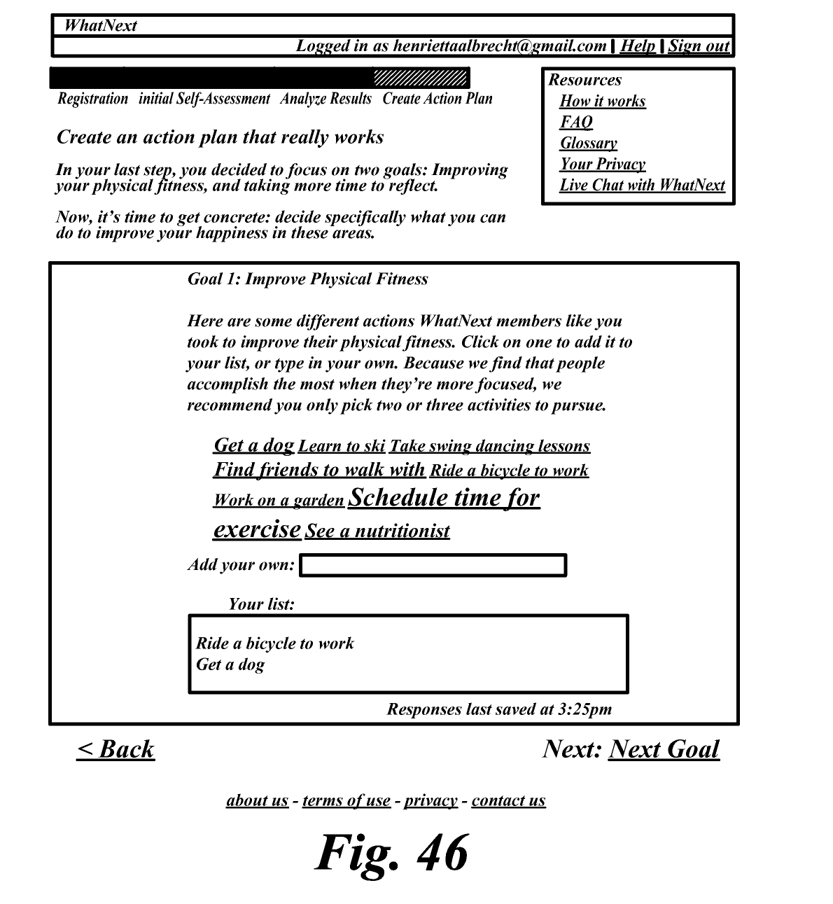

FIG. 46 shows a screenshot of a sample action plan. The action plan asks a member to provide goals that they can work toward to meet a particular area of need. This screen includes a list of what others have done and an area for a member to enter their individual goals.

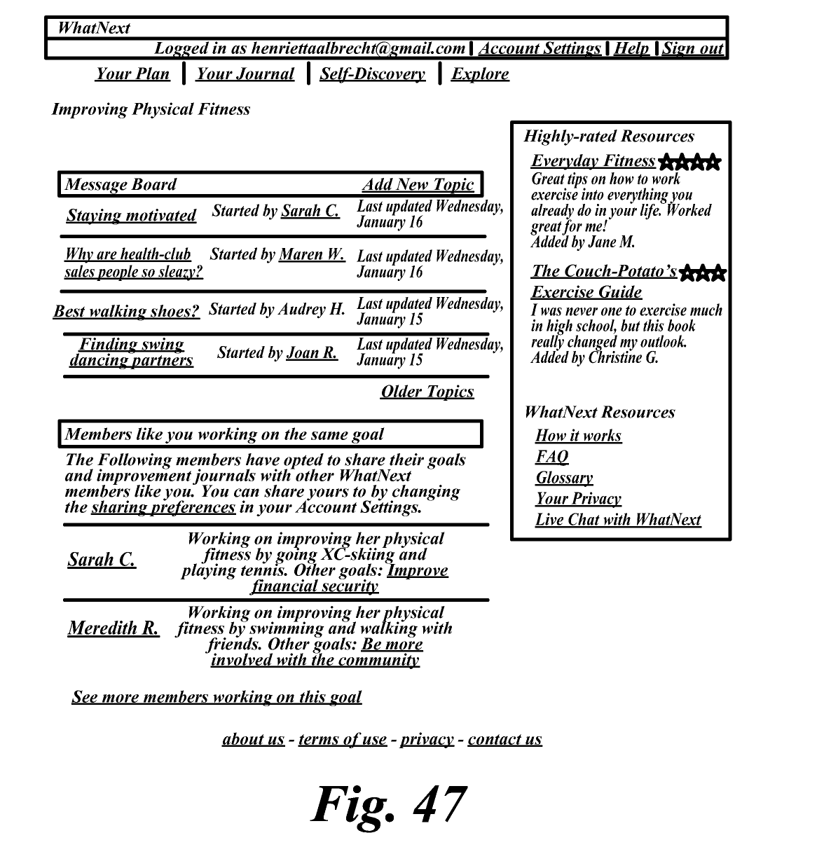

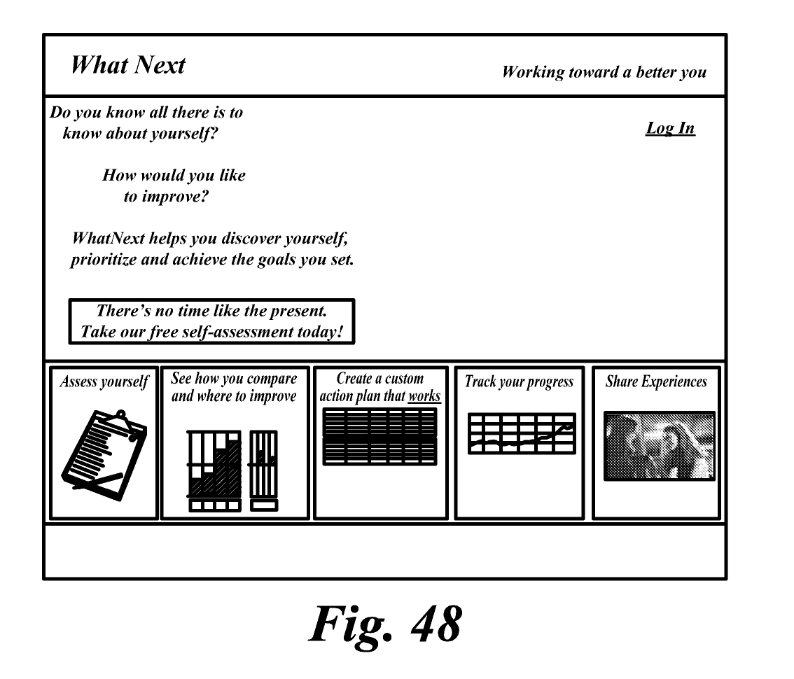

FIG. 47 shows a screenshot of sample community forums. In the community forum area a member has access to the complete message board, and access to member's journal's who are working on the same goal. The member has the ability to share their goals and improvement journals with other members. The screen also provides recommended resources on a particular topic.

FIG. 48 shows a screenshot of a sample advertisement and overview of the system and method.

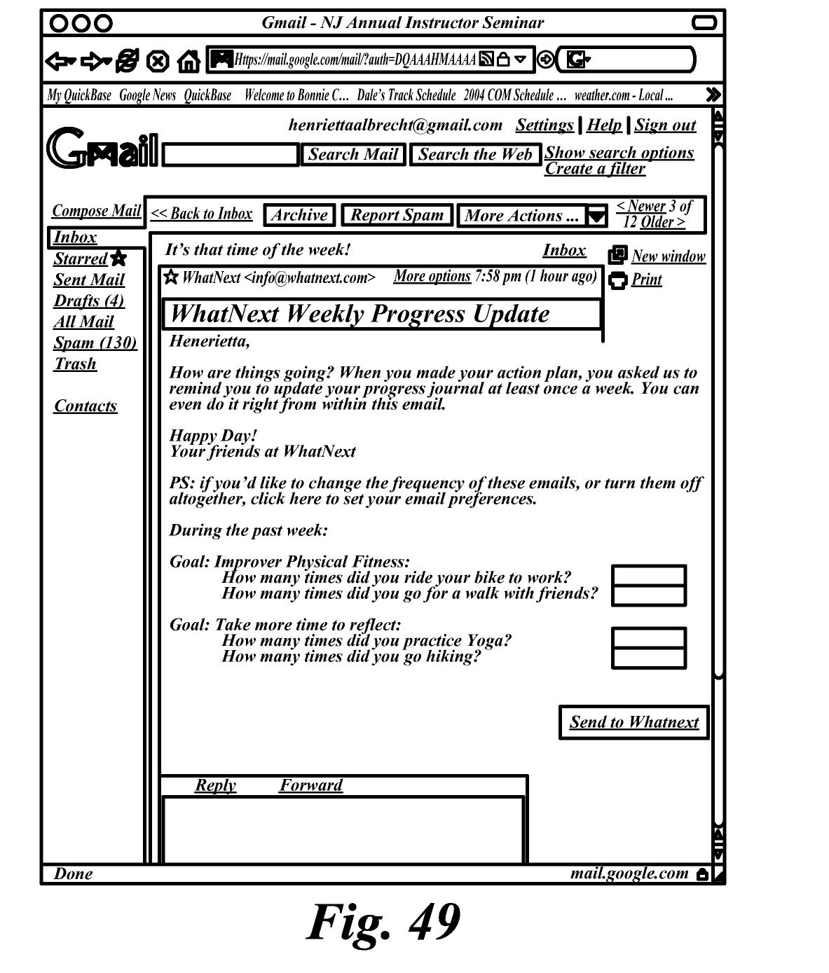

FIG. 49 shows a sample email that is sent to a member to remind them of their goals and action plan. It is an email that asks the member to update their journal and allows the member to update directly from the email.

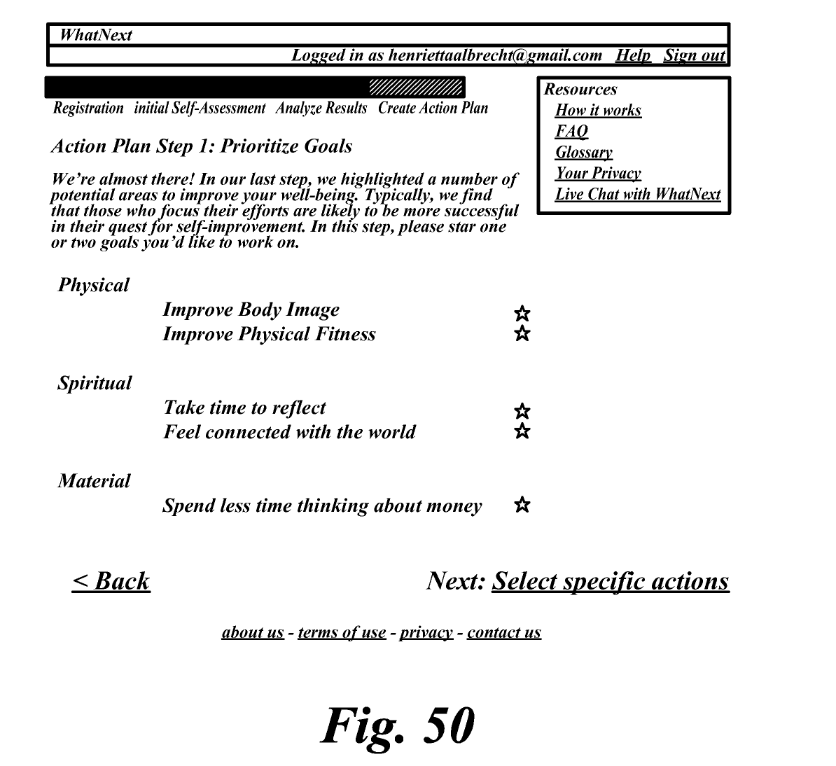

FIG. 50 shows a screenshot of a prioritizing goals screen. This screen allows a member to review potential areas to improve well being. It then allows the member to choose at least one goal to work on.

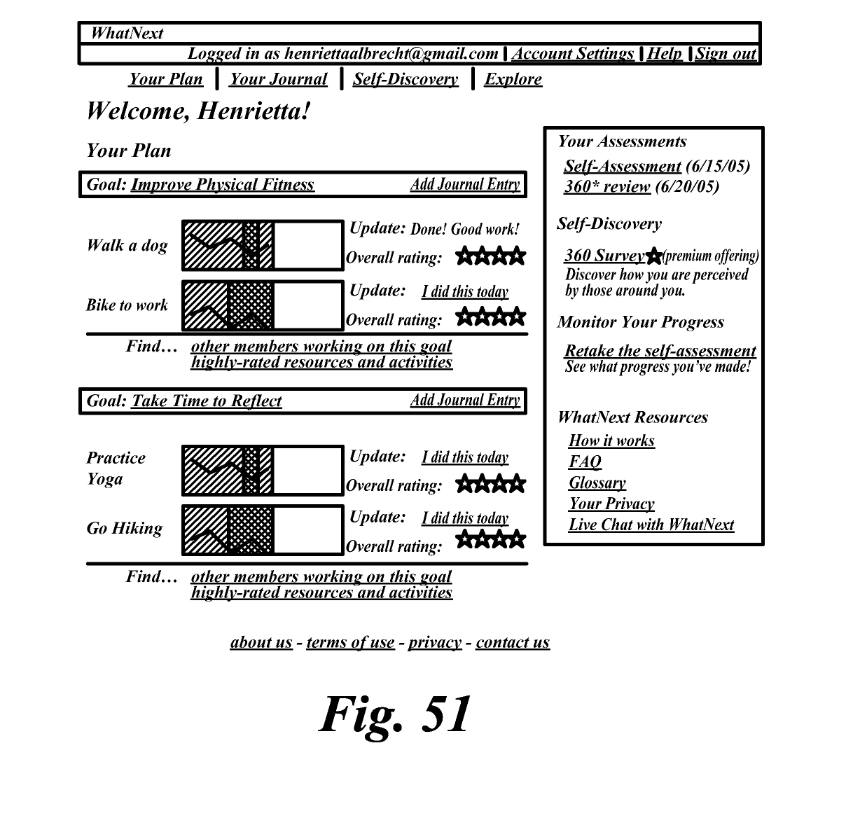

FIG. 51 shows a screenshot of a plan developed for a member. The screenshot includes a plan having a plurality of goals and a method to update the progress. It also includes links to your assessments, self discovery; monitor your progress, and additional resources.

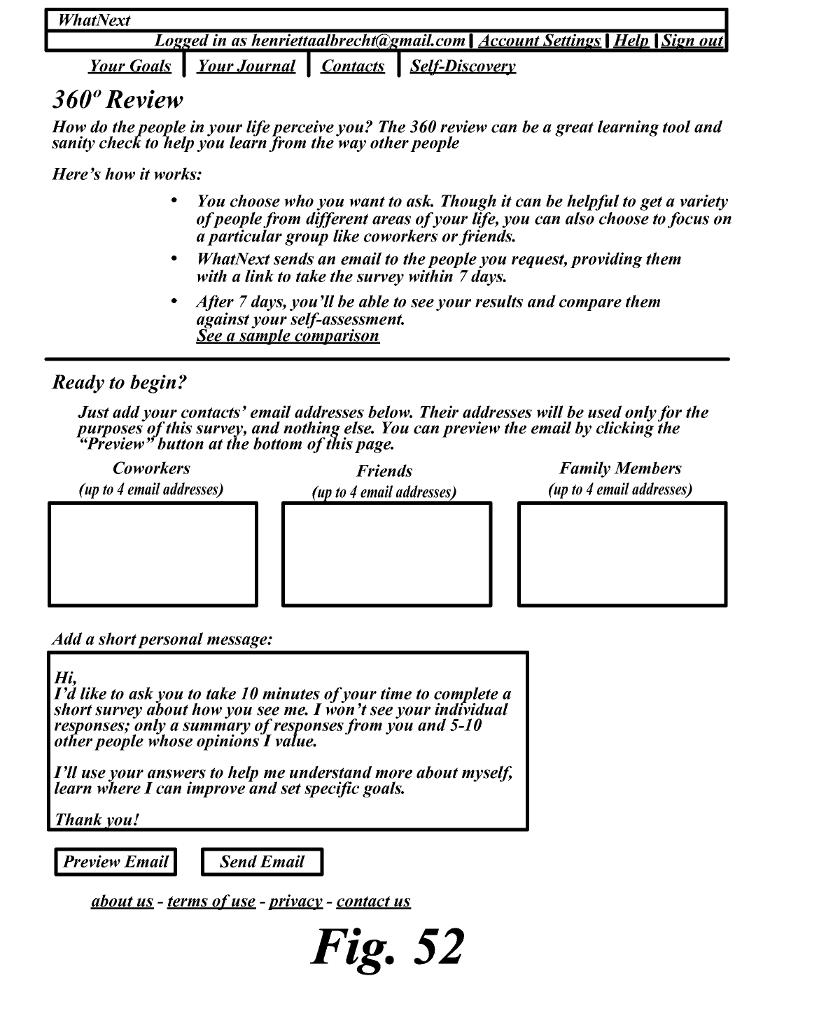

FIG. 52 shows a screenshot of a 360° review. The 360° review is sent to three peers and those peers complete a survey about you. Those results allow for a comparison between how you view yourself and how other view you. It can also provide a more accurate set of data to produce recommendations.

While the particular embodiments have been illustrated and described. Other modifications and changes varied to fit particular operating requirements and environments can be apparent to those skilled in the art, the invention is not considered limited to the example chosen for purposes of disclosure, and covers all changes and modifications which do not constitute departures from the true spirit and scope of this invention. Instead, embodiments of the invention should be determined entirely by reference to the claims that follow.

### APPENDIX

1. Studies having “X” denotes positive causal correlation of behavior dimension vs. resultant outcome. Examples show 28 to 38 behavior dimensions are positively correlated with performance and productivity and 12 of 38 behavior dimensions affect attrition from and/or intention to leave an organization. Five behavior dimensions positively affect health care costs and ten behavior dimensions positively affect well-being.

| | | | | | | | | |
|---|---|---|---|---|---|---|---|---|
| | | | Claims/ | Health | | | | Attrition/ |
| | Performance/ | Absentee- | Injuries/ | Care | Job | Well- | Overall | Intention to |
| Dimension | Productivity | ism | Accidents | Costs | Satisfaction | Being | Health | Leave |

Physical

| | | | | | | | |
|---|---|---|---|---|---|---|---|
| Overall Physical Health | X | X | X | [symbol] | | | |
| Sleep | X | X | X | | | | |
| Energy Level | X | | | | | | |
| Nutrition | X | | | X | X | X | |
| Exercise & Fitness | | X | | X | | | |
| Sexuality | | | | | | X | |
| Positive Health Indicators | X | | | | X | X | X |
| Managing Addiction | X | X | X | X | | | |

Emotional

| | | | | | | | | |
|---|---|---|---|---|---|---|---|---|
| Self-Acceptance | X | | | | | | | |
| Managing Stress & Anxiety | X | | | X | | X | X | |
| Managing Depression | X | | | X | X | | | X |
| Self-Control | X | X | X | | | | | |
| Autonomy | X | | | | X | X | | X |
| Managing Fears & Phobias | X | | | | | | | |
| Positive Relationships | X | | | | X | X | | |

Actualization/Reaching Potential

| | | | | | |
|---|---|---|---|---|---|
| Life Meaning | | | | X | |
| Growth | X | | X | | X |
| Flow | X | | | | |
| Savor | | | | X | |
| Altruism | X | | X | X | |
| Mindfulness | | X | | X | |

Capacity for Change

| | | | | | |
|---|---|---|---|---|---|
| Making & Keeping Commitments | | | | X | |
| Readiness | X | | | X | |
| Resilience | X | | X | | X |
| Self-Efficacy | X | X | X | | X |
| Openness & Optimism | | | | X | |

Work Well-Being

| | | | | |
|---|---|---|---|---|
| Basic Job Satisfaction | X | X | [symbol] | X |
| Energizing | X | | | X |
| Balance (Work/Life Balance) | X | X | | X |
| Work Growth | X | | | |
| Resources & Support | X | X | | X |
| Sense of Team | X | X | | |
| Work Meaning | X | X | | X |
| Fit with Culture | X | | | X |
| Belief In Company | | | X | |
| Square Deal | X | X | | X |
| Dream Job | | | | |
| External Factors | | | | |

Overall Well-Being

| | | | | | | | |
|---|---|---|---|---|---|---|---|
| Overall Well-Being (from Lyubomirsky, King, | X | X | X | X | X | X | X |
| Diener. 2005) | | | | | | | |

2. Software code for Limeade Goal Assistant:

/* ------------------------------------------------------------------------------

| Limeade.GoalAssistant | |
|---|---|
| (c) Copyright by Limeade, Inc. 2006-2007 | |
| Application: Limeade Platform | |
| Version: 2.0 | |
| COPYRIGHT NOTICE: | See attached License Agreement. |

| All rights reserved. Reproduction or transmission in whole or in part, in | | | |
|---|---|---|---|
| any form or by any means, electronic, mechanical or otherwise, is prohibited | | | |
| without the prior written consent of the copyright owner. | | | |
| Developer: | | | |
| Erick Rivas - EAR - (http://www.erickrivas.net/) | | | |
| History: | | | |
| Date | Initials | Change | Description |
| ------------------------------------------------------------------------------- | | | |
| 5.22.06 | EAR | Added | GoalAssistant |
| 6.23.06 | EAR | Added | RecommendationTemplates |

------------------------------------------------------------------------------- */

using System;
using System.Collections;
using System.IO;
using System.Text;
using System.Data;
using System.Data.SqlClient;
using Sysrem.Collections.Generic;
using System.Text.RegularExpressions;
namespace Limeade
{

/// <summary>
/// Goal Assistant Exception
/// </summary>
public class GoalAssistantException : Exception
{

/// <summary>
/// Goal Assistant Exception
/// </summary>
/// <param name=“msg”>Error message</param>
public GoalAssistanttxception(string msg) : base(msg)
{
}

}
public class MemberScore
{

public int ScoreType;
public string ScoreName;
public string ScoreParent;
public string ScoreValue;

public int ScoreTotal;
public int NumAnswers;

public string Conditions;
public string Actions;
public MemberScore( ) ( )
public mt Compare(object 01, object 02)
{
}

}
public class Goal
{

public int GoalId;
public string Membername;
public string GoalName;

public string GoalIcon;

public bool IsCommunityGoal;
public bool IsTopThree;
public bool IsAmount;
public string Amount;
public string AmountUnits;
public string AmountFrequency;
public string Deadline; // use proper date/time type
public int UseCount;
public string Rating;
public string Keywords;

public string TrackingToolName;
public string TrackingToolParams;
public int TrackingToolId;

public string ForumName;
public int ForumId;
public int BlogId;
public int FolderId;
public int GalleryId;
public string Podcast;
public Goal( ) ( )

}
public class Activity
{

public int ActivityId;
public int ParentActivityId;
public int GoalId;
public string Membername;
public string ActivityName;
public bool IsCommunityActivity;
public bool IsSponsoredActivity;
public int ActivityProviderId;
public string ActivityProviderName;
public bool IsAmount;
public string Amount;
public string AmountUnits;
public string AmountFrequency;
public string Deadline; // use proper date/time type
public int UseCount;
public string Rating;
public string Keywords;
public string HTMLSnippet;
public Activity( ) ( )

}
public enum LimeadeMicroformat
{

LIMEADE_FORMAT_STARTRANGE = 0,
AreaCSV = 10,
AssessmentCSV = 11,
NemberScoresCSV = 12,
GoalCSV = 14,
ActivityCSV =15,
GoalAndActivityCSV =16,
_LimeadeXML = −1

}
public enum RecommendationTemplateAlgorithm
{

RECOMMENDATION_TEMPLATE_STARTRANGE =0,
Guest = 1,
RegisteredMember = 2,
AdvancedMember = 3,
PaidMember = 4,
Expert = 5,
PowerMember = 6,
Employee = 7,
ENTERPRISE_TEMPLATE_STARTRANGE = 300777,
MICROSOFT_ENTERPRISE_HR_DEMO = 300778,
OEM_TEMPLATE_STARTRANGE = 400777,
_360CXMap = −1

}
public enum RecommendationTemplateVariable
{

RECOMMENDATION_VAR_STARTRANGE = 0,
Answers = 1,
Scores = 2,
Rating = 3,
Popularity = 4,
ProfileFields = 5,
AccountSettings = 6,
HistoricalData = 7,
XmlChartData = 8,
Journal = 9,
Blogs = 10,
OutlookRules = 11,
Photos = 12,
CommunityForumPosts = 13,
Podcasts = 14,
RSSFeeds = 15,
ENTERPRISE_VAR_STARTRANGE = 100777,
OEM_VAR_STARTRANGE = 200777,
_360CXMap = −1

}
/// <summary>
/// </summary>
public class GoalAssistant

{
public GoalAssistant( ) ( )

public bool AddTopRecommendationsToPlan(string u)
{

return RecommendGoals(u, true)

}

public bool RecommendGoals(string u)

{

return RecommendGoals(u, false);

}

/// <summary>
// RecommendGoals based on CommunityGoals X Member u Score table
/// </summary>

public bool RecommendGoals(string u, bool addtoprecommendationstoplan)

{
}
static int Search(string input, string regularexpression)
{
}
/// <summary>
// add recommended Goals to Member u by searching for Community Goals that have

same/similar keywords

/// </summary>
public bool RecommendRelatedGoals(string u, string keywords, bool addtoplan)
{
}
/// <summary>
// ImproveRecommendations for Member u Goals based on Assessment Model X Recommendation

Template Algorithm X RecommendationTemplateVariables [ ]

/// <summary>
public bool ImproveRecommendations(string u, RecommendationTemplateAlgorithm rta,

RecommendationTemplateVariable [ ] rtv )

{
}
/// <summary>
// ImproveRecommendations for Member u goalname Activities based on Assessment Model X

Recommendation Template Algorithm X RecommendationTemplateVariables [ ]

/// <summary>
public bool ImproveRecommendations (string u, string goalname,

RecommendationTemplateAlgorithm rta, RecommendationTemplareVariable [ ] rtv)

{
}

}

}

3. Examples of Questionnaires vs. Behavior Dimension:

| | |
|---|---|
| Blood pressure | What is your blood pressure? |
| | Have you ever been told that you have high blood |
| | pressure? |
| Body weight | How much do you weigh? |
| | How tall are you? |
| Cholesterol | What is your cholesterol level? |
| | Have you ever been told that you have high |
| | cholesterol? |
| HDL (good | What is your HDL or good cholesterol level? |
| cholesterol) | Have you ever been told that your “good” |
| | cholesterol level is [symbol] |
| Alcohol | How many alcholic drinks do you typically hate every |
| | day? |
| Existing medical | Do you currently have any of the following? |
| condition | In the past, have you had any of the following? |
| Stress | Which of the following best describes your current |
| | level of s [symbol] |
| Smoking | Do you smoke cigarettes or a pipe? |
| Exercise | How often do you exercise? |
| Absenteeism | Approximately how many days did you miss from |
| | work last? |
| Seat Belt | How often do you wear your seat belt? |
| Health perception | Overall, how would you describe your health? |
| Demographics | What is your sex? |

The embodiments of the invention in which an exclusive property or privilege is claimed are defined as follows:

1. A method to determine the health of employees of an organization comprising:
recruiting at least a portion of the employees to participate in a health assessment program;
providing the health assessment program to the employees, the health assessment program comprising a well-being category having a parameter defined by at least one of a physical, emotional, actualization, capacity for change and work well-being;
engaging the at least portion of employees with surveys applicable to at least one of the well-being categories;
analyzing the surveys; and based on the analysis; and
presenting an employee with behavior change tools and resources.
